(19) United States
(12) Patent Application Publication
(10) Pub. No.: US 2003/0206910 A1
(43) Pub. Date: Nov. 6, 2003

(54) Poloxamer and poloxamine compositions for nucleic acid delivery

(76) Inventors: Francois Nicol, Menlo Park, CA (US); Jijun Wang, Pearland, TX (US); Michael E. Coleman, The Woodlands, TX (US); Fiona MacLaughlin, Belfast (GB); Alain Rolland, San Diego, CA (US)

Correspondence Address: Patent - LA, Perkins Coie LLP, P.O. Box 1208, Seattle, WA, 98111-1208, US

(21) Appl. No.: 10/234,405

(22) Filed: Sep. 3, 2002

Related U.S. Application Data
(63) Continuation-in-part of application No. PCT/US01/06831, filed on Mar. 2, 2001.
(60) Provisional application No. 60/187,236, filed on Mar. 3, 2000.
(60) Provisional application No. 60/242,277, filed on Oct. 20, 2000.

(51) Int. Cl. A61K 48/00

(52) U.S. Cl. 424/178.1; 514/44

(57) ABSTRACT

The invention features selected polymers such as poloxamer 188 (Pluronic® F68), poloxamer 237 (Pluronic® F87), poloxamer 401 (Pluronic® L121), poloxamer 338 (Pluronic® F108), Poloxamer 124 (Pluronic® L44), Poloxamer 184 (L-64), and poloxamines (Tetronics® 904, 908, 1107, and 90R4) that enhance expression from nucleic acids when administered into an organism, in particular to muscle, and further provides selected poloxamer formulations for delivery of nucleic acids to the liver.

### INTRODUCTION

This invention relates to compositions and methods for the introduction of a nucleic acid molecule into a cell, preferably by a pulse voltage delivery method, for the expression of a protein, peptide, antisense RNA, ribozyme, or polypeptide. It is useful for in vitro transfections, in vivo gene therapy, administration of therapeutic proteins, peptides and polypeptides, and vaccination. This is the U.S. National Application of and a Continuation-In-Part of International Patent Application No. PCT/US01/06831, which claims priority to U.S. Provisional Application No. 60/187,236 filed Mar. 3, 2000 and U.S. Provisional Application No. 60/242,277 filed Oct. 20, 2000, which are all hereby incorporated by reference, including any drawings, as is fully set forth herein.

### BACKGROUND OF THE INVENTION

Gene therapy is a major area of research in drug development. A technological barrier to commercialization of gene therapy, however, is the need for practical and effective gene delivery methods. A problem of gene injection by conventional needle-syringe methods is that genetic material must be injected in large quantities into the target.

To overcome the problem of degradation of plasmids and enhance the efficiency of gene transfection, cationic condensing agents (such as polybrene, dendrimers, chitosan, lipids, and peptides) have been developed to protect pDNA by condensing it through electrostatic interaction. A. P. Rolland, From genes to gene medicines: recent advances in nonviral gene delivery, review in Therapeutic drug carrier systems, 15(2):143-198 (1998). However, the use of condensed plasmid particles for transfection of a large number of muscle cells in vivo has not been successful, as compared directly to “naked” DNA. Wolff, J. A., et al., J. Cell Sci., 103, 1249, 1992.

Biodegradable microspheres have been successfully used to deliver drugs at a controlled rate to specific tissues. U.S. Pat. No. 5,160,745 to DeLuca et al., issued Nov. 3, 1992, discloses a microencapsulated biologically active macromolecular agent. The microencapsulant is a biodegradable vinyl derivative. The use of microsphere encapsulation has been extended to use in gene delivery. WO0078357, Chen, W. et al, disclosed matrices, films, gels and hydrogels which include hyaluronic acid (HA) derivatized with a dihydrazide and crosslinked to a nucleic acid forming slow release microspheres. WO9524929, Boekelheide, K. et al., disclosed encapsulation of genes in a matrix preferably in the form of a microparticle such as a microsphere, microcapsule, a film, an implant, or a coating on a device such as a stent. U.S. Pat. No. 6,048,551, Beer, S. et al. disclosed a controlled release gene delivery system utilizing poly(lactide-co-glycolide) (PLGA), hydroxypropylmethyl cellulose phthalate, cellulose acetate phthalate, and the Ludragit R, L, and E series of polymers and copolymer microspheres to encapsulate the gene vector. Luo D et al. Pharm Res August 1999;16(8):1300-8, reported the characterization of systems for controlled delivery of DNA from implantable polymer matrices (EVAc: poly (ethylene-co-vinyl acetate)) and injectable microspheres (PLGA and PLA: poly (D, L-lactide-co-glycolide) copolymer and poly (L-lactide), respectively). Despite their promise, microspheres can pose manufacturing difficulties and can adversely constrain the release of DNA in vivo, particularly in muscle tissue.

The use of cationic polymers derived from poloxamer or poloamines has been disclosed. The requirement for cationie and polycationic elements is in keeping with the conventional wisdom that a composition for gene delivery must be able to condense DNA into a particle in order to be effective. Thus, U.S. Pat. No. 5,656,611, Alakhov et al., discloses polycationic complexes of polynucleotides covalently linked to poloxamer or poloamines having integral polycationic segments for gene delivery. Similarly, WO0051645, S. Davis et al., discloses positively charged poloxamers and poloxamines for gene delivery.

The use of protective interactive noncondensing (PINC) polymers, such as poly(N-vinyl pyrrolidone) (PVP), poly(viny alcohol) (PVA) and poloxamer 407 to enhance the delivery of plasmids to rat skeletal muscle has been disclosed in U.S. patent application Ser. No. 08/372,213, now U.S. Pat. No. 6,040,190, and Ser. No. 08/798,274, both of which are incorporated herein by reference in their entirety, including any drawings.

Injection by electroporation is a technique that involves the application of a pulsed electric field to create transient pores in the cellular membrane without causing permanent damage to the cell and thereby allows for the introduction of exogenous molecules. PINC formulations for electroporation are described in U.S. patent application Ser. No. 09/322,602, which is incorporated herein by reference in its entirety, including any drawings. By adjusting the electrical pulse generated by an electroporetic system, nucleic acid molecules can find their way in the cell through passageways or pores that are created during the procedure.

Despite these recent advances there remains need for additional and improved formulated nucleic acid compositions and methods of administering the same for gene therapy.

### SUMMARY OF THE INVENTION

This invention features compositions and methods for enhancing the administration of nucleic acids and uptake thereof by an organism. An efficient strategy for enhancing nucleic acid delivery in vivo is to protect the nucleic acid from degradation, thereby maintaining the administered nucleic acid at the target site in order to further increase its cellular uptake (i.e., incorporation into cells). Also, for in vitro administration, increasing the effective concentration of the nucleic acid at the cell surface should increase the efficiency of transfection. The compositions of the present invention which are used to administer nucleic acid, preferably by pulse voltage delivery, include a compound which protects the nucleic acid and/or prolongs the localized bioavailability of the nucleic acid when administered to an organism in vivo, or in vitro in cell culture. Furthermore, the present invention also allows for treatment of diseases, vaccination, and treatment of muscle disorders and serum protein deficiencies.

In preferred embodiments, the invention features a number of new PINC polymers, including poloxamers 124, 188, 237, 338, and 401 and poloxamines (Tetronics®) to enhance delivery of genes to muscle cells after their direct intramuscular administration. The poloxamers of the invention reseal the myofiber membranes and also permeabilize them to some extent, or otherwise enhance plasmid uptake or expression. The polaxamer formulations are also useful in enhancing the resulting immune response to plasmid encoded antigens.

Thus, in one aspect, the invention features a composition for delivery of a nucleic acid molecule to a cell. The composition, also referred to herein as a formulated nucleic acid molecule, includes (a) a protective, interactive, non-condensing compound selected from the group consisting of poloxamer 188, poloxamer 124, and poloxamer 401, and (b) a nucleic acid molecule that includes a sequence encoding a gene product.

Compositions and methods are provided for deleivery of poloxamer formulations of nucleic acids to the liver. In one embodiment, 2-8% solutions of poloxamers L-44 and L-64 are provided for increasing the delivery of nucleic acids to the liver by either bolus injection or slow infusion via the hepatic artery, hepatic portal vein, hepatic vein or bile duct.

The PINC enhances the delivery of the nucleic acid molecule to mammalian cells in vivo, and preferably the nucleic acid molecule includes a coding sequence for a gene product to be expressed in the cell. In many cases, the relevant gene product is a polypeptide or protein. Preferably the PINC is used under conditions so that the PINC does not form a gel, or so that no gel form is present at the time of administration at about 30-40° C. Thus, in these compositions, the PINC is present at a concentration of 15% (w/v) or less. In certain preferred embodiments, the PINC concentration is still less, for example, 10% or less, 8% or less, 5% or less, or 1% or less. Thus, these compositions differ in compound concentration and functional effect from uses of these or similar compounds in which the compounds are used at higher concentrations, for example in the ethylene glycol mediated transfection of plant protoplasts, or the formation of gels for drug or nucleic acid delivery. In general, the PINCs are not in gel form in the conditions in which they are used as PINCs, though certain of the compounds may form gels under some conditions.

In another aspect, the invention provides a method of administering to a mammal a composition for delivery of a nucleic acid molecule to a cell. The method involves the step of introducing, preferably by injection, a composition of the invention into a tissue (e.g., muscle or a tumor) or interstitial space of a mammal.

Administration as used herein refers to the route of introducing the compositions of the invention into the body of cells or organisms. Administration includes the use of electroporetic methods as provided by a pulse voltage device to targeted areas of the mammalian body such as the muscle cells and the lymphatic cells in regions such as the lymph nodes. Administration also includes intradermal, intra-tumoral and subcutaneous administration. Another aspect provides a method for treating a mammalian condition or disease, preferably cancer. The method involves the step of administering to a mammal suffering from the condition or disease a therapeutically effective amount of a composition of the invention. A “therapeutically effective amount” of a composition is an amount that is sufficient to cause at least temporary relief or improvement in a symptom or indication of a disease or condition. Thus, the amount is also sufficient to cause a pharmacological effect. The amount of the composition need not cause permanent improvement or improvement of all symptoms or indications.

The invention also features a method of making the compositions of the invention by combining the PINC compound and the nucleic acid molecule.

In yet another aspect, the invention also features a method for delivering a nucleic acid molecule to an organism, preferably a plant or a mammal, more preferably a human. The method involves the step of providing a composition of the invention to the cells of the organism by use of a device configured and arranged to cause pulse voltage delivery of the composition.

In preferred embodiments, the method results in an antibody response, an immune response, a humoral immune response, a T-cell mediated immune response, a prophylactic immune response, or a therapeutic immune response.

In preferred embodiments the device for delivering is an electroporation device that delivers the composition of the invention to the cell by pulse voltage and/or the delivering of the composition of the invention involves subjecting the cells to an electric field.

The present invention also features a kit. The kit includes a container for providing a composition of the invention and either (i) a pulse voltage device for delivering the composition of the invention to cells of an organism, wherein the pulse voltage device is capable of being combined with the container, or (ii) instructions explaining how to deliver the composition of the invention with the pulse voltage device.

Thus the “container” can include instructions furnished to allow one of ordinary skill in the art to make compositions of the invention. The instructions will furnish steps to make the compounds used for formulating nucleic acid molecules. Additionally, the instructions will include methods for testing compositions of the invention that entail establishing if the nucleic acid molecules are damaged upon injection after electroporation. The kit may also include notification of an FDA approved use and instructions.

A method for making a kit of the invention is also provided. The method involves the steps of combining a container for providing a composition of the invention with either (i) a pulse voltage device for delivering the composition of the invention to the cells of an organism, wherein the pulse voltage device is capable of being combined with the container, or (ii) instructions explaining how to deliver the composition of the invention with the pulse voltage device.

The invention also provides a method of treating a mammal suffering from cancer or an infectious disease. The method involves the step of providing a composition of the invention to cells of the mammal by use of a device configured and arranged to provide pulse voltage delivery of a composition of the invention to cells of the mammal, wherein the molecule encodes a cancer antigen or an antigen for the infectious disease.

In preferred embodiments the cancer antigen is MAGE 1, and the cancer is melanoma and/or the infectious disease antigen is HBV core antigen, and the infectious disease is chronic hepatitis.

As noted above, the compositions of the present invention that are used to administer nucleic acid, preferably by pulse voltage delivery, include a compound which protects the nucleic acid and/or prolongs the localized bioavailability of the nucleic acid when administered to an organism in vivo, or in vitro in cell culture.

As the compositions are useful for delivery of a nucleic acid molecule to cells in vivo, in a related aspect the invention provides a composition at an in vivo site of administration. In particular this includes at an in vivo site in a mammal.

The summary of the invention described above is not limiting and other and further objects, features and advantages of the invention will be apparent from the following detailed description of the presently preferred embodiments of the invention and from the claims.

### BRIEF DESCRIPTION OF THE DRAWINGS

FIG. 1 shows the effect of polymer concentration on the luciferase reporter gene expression in CD-1 mice after intramuscular (IM) injection of pDNA in poloxamer 188 formulations. A and B were parallel experiments with 10 microgram DNA and 30 microgram DNA/muscle, respectively. Results are reported as mean±SEM (n=10).

FIG. 2 shows the histology of mice tibialis muscles for Green Fluorescent Protein (GFP) at day 5 after 1 IM injection pDNA in A) saline and B) 5% poloxamer 188 in saline. DNA injected per muscle was 10 microgram/10 microliter.

FIG. 3 shows the plasmid DNA dose-response of luciferase expression at day 7 after injection of 10 microliters of formulation containing different concentrations of poloxamer 188. Results are reported as mean±SEM (n=10).

FIG. 4 shows the time-course of luciferase expression at day 7 after injection of CMV-luciferase (30 microgram of DNA/muscle). Results reported as mean±SEM (n=10).

FIG. 5 shows a comparison of selected poloxamer formulations on in vivo gene expression following im injection in mice.

FIG. 6 shows a comparison of 5% poloxamer 124, 5% poloxamer 188 and saline, 1 mg/ml SEAP plasmid, 25 μl injected into each mouse tibialis muscle (50 μg dose). Poloxamer 124 (L44) gave a 17-fold increase (p=0.03) in expression over saline at day 7 and 14-fold increase in area under the curve over 21 days. 5% poloxamer 188 (F68) was 6-fold better (p=0.15) than saline at day 7 and 6-fold better over 21 days. For both poloxamers, 1% and 10% concentrations were inferior to 5%.

FIG. 7A and B shows a dose response of SEAP plasmid 1-50 micrograms in 5% poloxamer 188 (F68) and 5% poloxamer 124 (L44).

FIG. 8 shows enhanced immune response using poloxamer 401 (L121). Mice were injected either intramuscularily or intradermally with the indicated formulations of β-gal expressing plasmid DNA and tested for antibody formation to β-gal protein.

FIG. 9 shows enhanced CTL activity following IM administration of poloxamer 401 (L121)/plasmid formulations.

FIG. 10 shows the stability profile of hDel-1 encoding plasmid DNA in 5% poloxamer 188 (F68) at 37° C.

FIG. 11 shows the expression of SEAP from plasmid DNA formulated in saline versus 5% poloxamer 188 (F68), and various poloxamine formulations.

FIG. 12 shows the results of the DNase protection assay comparing saline with 5% poloxamer 188 (F68) or 6 mg/ml Poly-L-glutamate. Panel A represents a DNA in saline formulation; Panel B represents DNA formulated in 5% poloxamer 188 (F68); Panel C represents DNA formulated in 6 mg/ml poly-L-glutamate. Lane A, negative control of plasmid DNA without DNase; lane B, positive control of plasmid DNA and DNase mixed 1:1; lane C, DNase diluted 1:1; lane D, DNase diluted 1:10; lane E, DNase diluted 1:100; lane F, DNase diluted 1:1000; lane G, DNase diluted 1:10000. In saline, DNase at 1:100 is able to abolish the lower band of supercoiled plasmid in addition to degradation of the DNA resulting in a smear of different molecular weights on the gel. In contrast, both poly-L-glutamate and poloxamer 188 (F68) were able to confer protection from DNase degradation.

FIG. 13 shows the importance of polymer formulation process parameters on reporter gene expression.

FIG. 14 shows expression of mDel1 in tibialis anterior muscles of mice after delivery of plasmid DNA in a 5% poloxamer 188 (F68) formulation with and without electroporation.

FIG. 15 shows the biological effect of hDel-1 expression on capillary density in normoxic mouse skeletal muscle after delivery of plasmid DNA in a 5% poloxamer 188 (F68) formulation.

FIG. 16 shows the enhanced expression of hIL-12 when delivered to the lung intravenously in 1-12% poloxamer 188 (F68) formulations compared with DOTMA/Chol.

FIG. 17 shows the enhanced expression of hIL-12 when delivered by hepatic artery delivery in a 5% poloxamer 188 (F68) formulation compared with DOTMA/Chol, saline or mannitol.

FIG. 18 shows the relationship of polyoxyethylene to polyoxypropylene ratio in different PLURONIC® type poloxamer polymers and the non-proprietary nomenclature of the PLURONIC® polymers used.

FIG. 19 further depicts the nomenclature and chemical characteristics of the poloxamers employed.

FIG. 20 shows the relative expression levels from a human IL-12 plasmid delivered by bolus injection to the hepatic artery with either L-44 or F68 at 2 and 4%.

FIG. 21 shows the relative expression levels from a luciferase encoding plasmid delivered by slow infusion with L-44 at 4 and 8% compared against saline.

The drawings are not necessarily to scale. Certain features of the invention may be exaggerated in scale or shown in schematic form in the interest of clarity and conciseness.

### DETAILED DESCRIPTION OF THE PREFERRED EMBODIMENTS

The delivery and expression of sequences encoded on a vector in eukaryotic cells, particularly in vivo in a mammal, depend on a variety of factors including transfection effeciency and lifetime of the coding sequence within the transfected cell. Thus, a number of methods are reported for accomplishing such delivery.

By “delivery” or “delivering” is meant transportation of nucleic acid molecules to desired cells or any cells. The nucleic acid molecules may be delivered to multiple cell lines, including the desired target. Delivery results in the nucleic acid molecules coming in contact with the cell surface, cell membrane, cell endosome, within the cell membrane, nucleus or within the nucleus, or any other desired area of the cell from which transfection can occur within a variety of cell lines which can include but are not limited to; tumor cells, epithelial cells, Langerhan cells, Langhans' cells, littoral cells, keratinocytes, dendritic cells, macrophage cells, kupffer cells, muscle cells, lymphocytes and lymph nodes. Preferably, the composition of the invention is delivered to the cells by electroporation and the nucleic acid molecule component is not significantly sheared upon delivery, nor is cell viability directly effected by the pulse voltage delivery process.

By “nucleic acid” is meant both RNA and DNA including: cDNA, genomic DNA, plasmid DNA or condensed nucleic acid, nucleic acid formulated with cationic lipids, nucleic acid formulated with peptides, cationic polymers, RNA or mRNA. In a preferred embodiment, the nucleic acid administered is plasmid DNA that includes a “vector”. The nucleic acid can be, but is not limited to, a plasmid DNA vector with a eukaryotic promoter which expresses a protein with potential therapeutic action, such as, for example; hGH, VEGF, DEL-1, EPO, IGF-I, TPO, Factor IX, IFN-α, IFN-β, IL-2, IL-12, or the like.

As used herein, the term a “plasmid” refers to a construct made up of genetic material (i.e., nucleic acids). It includes genetic elements arranged such that an inserted coding sequence can be transcribed in eukaryotic cells. Also, while the plasmid may include a sequence from a viral nucleic acid, such viral sequence preferably does not cause the incorporation of the plasmid into a viral particle, and the plasmid is therefore a non-viral vector. Preferably a plasmid is a closed circular DNA molecule.

The term “vector” as used herein refers to a construction including genetic material designed to direct transformation of a targeted cell. A vector contains multiple genetic material, preferably contiguous fragments of DNA or RNA, positionally and sequentially oriented with other necessary elements such that the nucleic acid can be transcribed and when necessary translated in the transfected cells. The “vector” preferably is a nucleic acid molecule incorporating sequences encoding therapeutic product(s) as well as, various regulatory elements for transcription, translation, transcript stability, replication, and other functions as are known in the art. The vector preferably allows for production of a product encoded for by a nucleic acid sequence contained in the vector. For example, expression of a particular growth factor protein encoded by a particular gene. The vector may be a DNA vector or a viral vector. A “DNA vector” is a vector whose native form is a DNA molecule. A “viral vector” is a vector whose native form is as the genomic material of a viral particle.

“Post-translational processing” means modifications made to the expressed gene product. These may include addition of side chains such as carbohydrates, lipids, inorganic or organic compounds, the cleavage of targeting signals or propeptide elements, as well as the positioning of the gene product in a particular compartment of the cell such as the mitochondria, nucleus, or membranes. The vector may include one or more genes in a linear or circularized configuration. The vector may also include a plasmid backbone or other elements involved in the production, manufacture, or analysis of a gene product.

In connection with the compounds and compositions of this invention, the term “protects” or “protective” refers to an effect of the interaction between such a compound and a nucleic acid such that the rate of degradation of the nucleic acid is decreased in a particular environment. Such degradation may be due a variety of different of factors, which specifically include the enzymatic action of a nuclease. The protective action may be provided in different ways, for example, by exclusion of the nuclease molecules or by exclusion of water.

The term “interactive” as used herein refers to the interaction between PINC's and nucleic acid molecules and/or cell wall components. Preferably, PINC polymers are capable of directly interacting with moieties of nucleic acid molecules and/or cell wall components. These interactions can facilitate transfection and/or transformation by, for example, helping associate the nucleic acid molecule-PINC complex closely with the cell wall as a result of biochemical interactions between the PINC and the cell wall and thereby mediate transfection. These interactions may also provide protection from nucleases by closely associating with the nucleic acid molecule.

The term “transfection” as used herein refers to the process of introducing DNA (e.g., formulated DNA expression vector) into a cell, thereby, allowing cellular transformation. Following entry into the cell, the transfected DNA may: (1) recombine with that of the host; (2) replicate independently as a plasmid or temperate phage; or (3) be maintained as an episome without replication prior to elimination.

As used herein, “transformation” relates to transient or permanent changes in the characteristics (expressed phenotype) of a cell induced by the uptake of a vector by that cell. Genetic material is introduced into a cell in a form where it expresses a specific gene product or alters the expression or effect of endogenous gene products. Transformation of the cell may be associated with production of a variety of gene products including protein and RNA. These products may function as intracellular or extracellular structural elements, ligands, hormones, neurotransmitters, growth regulating factors, enzymes, chemotoxins, serum proteins, receptors, carriers for small molecular weight compounds, drugs, immunomodulators, oncogenes, cytokines, tumor suppressors, toxins, tumor antigens, antigens, antisense inhibitors, triple strand forming inhibitors, ribozymes, or as a ligand recognizing specific structural determinants on cellular structures for the purpose of modifying their activity. This list is only an example and is not meant to be limiting.

In connection with the protective, interactive, non-condensing compounds for these compositions, the term “non-condensing” means that an associated nucleic acid is not condensed or collapsed by the interaction with the PINC at the concentrations used in the compositions. Thus, the PINCs differ in type and/or concentration from such condensing polymers. Examples of commonly used condensing polymers include polylysine, and cascade polymers (spherical polycations).

A “gene product” means products encoded by the vector. Examples of gene products include mRNA templates for translation, ribozymes, antisense RNA, proteins, glycoproteins, lipoproteins, phosphoproteins and polypeptides. The nucleic acid sequence encoding the gene product and/or the PINC compound may be associated with a targeting ligand to effect targeted delivery.

In connection with the association of a targeting ligand and a PINC, the term “bound with” means that the parts have an interaction with each other such that the physical association is thermodynamically favored, representing at least a local minimum in the free energy function for that association. Such interaction may involve covalent binding, or non-covalent interactions such as ionic, hydrogen bonding, van der Waals interactions, hydrophobic interactions, and combinations of such interactions.

“Uptake” means the translocation of the vector from the extracellular to intracellular compartments. This can involve receptor mediated processes, fusion with cell membranes, endocytosis, potocytosis, pinocytosis or other translocation mechanisms. The vector may be taken up.

“Intracellular trafficking” is the translocation of the vector within the cell from the point of uptake to the nucleus where expression of a gene product takes place. Alternatively, cytoplasmic expression of a nucleic acid construct utilizing, for example, a T7 polymerase system may be accomplished. Various steps in intracellular trafficking include endosomal release and compartmentalization of the vector within various extranuclear compartments, and nuclear entry.

“Endosomal release” is the egress of the vector from the endosome after endocytosis. This is an essential and potentially rate limiting step in the trafficking of vectors to the nucleus. A lytic peptide may be used to assist in this process.

A “lytic peptide” is a peptide which functions alone or in conjunction with another compound to penetrate the membrane of a cellular compartment, particularly a lysosomal or endosomal compartment, to allow the escape of the contents of that compartment to another cellular compartment such as the cytosolic and/or nuclear compartment.

“Compartmentalization” is the partitioning of vectors in different compartments within a defined extracellular or intracellular space. Significant extracellular compartments may include, for example, the vascular space, hair follicles, interstitial fluid, synovial fluid, cerebral spinal fluid, thyroid follicular fluid. Significant intracellular compartments may include endosome, potosome, lysosome, secondary lysosome, cytoplasmic granule, mitochondria, and the nucleus.

“Nuclear entry” is the translocation of the vector across the nuclear membrane into the nucleus where the gene may be transcribed.

“Elimination” is the removal or clearance of materials (vectors, transcripts, gene products) from a specific compartment over time. This term may be used to reflect elimination from the body, the vascular compartment, extracellular compartments, or intra-cellular compartments. Elimination includes translocation (excretion) from a particular compartment or biotransformation (degradation).

The PINC enhances the delivery of the nucleic acid molecule to mammalian cells in vivo, and preferably the nucleic acid molecule includes a coding sequence for a gene product to be expressed in the cell. In many cases, the relevant gene product is a polypeptide or protein. Preferably the PINC is used under conditions so that the PINC does not form a gel, or so that no gel form is present at the time of administration at about 30-40° C. Thus, in these compositions, the PINC is present at a concentration of 15% (w/v) or less. In certain preferred embodiments, the PINC concentration is still less, for example, 10% or less, 8% or less, 5% or less, or 1% or less. Thus, these compositions differ in compound concentration and functional effect from uses of these or similar compounds in which the compounds are used at higher concentrations, for example in the ethylene glycol mediated transfection of plant protoplasts, or the formation of gels for drug or nucleic acid delivery. In general, the PINCs are not in gel form in the conditions in which they are used as PINCs, though certain of the compounds may form gels under some conditions.

The term “pulse voltage device”, or “pulse voltage injection device” as used herein relates to an apparatus that is capable of causing or causes uptake of nucleic acid molecules into the cells of an organism by emitting a localized pulse of electricity to the cells, thereby causing the cell membrane to destabilize and result in the formation of passageways or pores in the cell membrane. It is understood that conventional devices of this type are calibrated to allow one of ordinary skill in the art to select and/or adjust the desired voltage amplitude and/or the duration of pulsed voltage and therefore it is expected that future devices that perform this function will also be calibrated in the same manner. The type of injection device is not considered a limiting aspect of the present invention. The primary importance of a pulse voltage device is, in fact, the capability of the device to deliver compositions of the invention into the cells of an organism. The pulse voltage injection device can include, for example, an electroporetic apparatus as described in U.S. Pat. No. 5,439,440, U.S. Pat. No. 5,704,908 or U.S. Pat. No. 5,702,384 or as published in PCT WO 96/12520, PCT WO 96/12006, PCT WO 95/19805, and PCT WO 97/07826, all of which are incorporated herein by reference in their entirety.

The term “apparatus” as used herein relates to the set of components that upon combination allow the delivery of compositions of the invention into the cells of an organism by pulse voltage delivery methods. The apparatus of the invention can be a combination of a syringe or syringes, various combinations of electrodes, devices which are useful for target selection by means such as optical fibers and video monitoring, and a generator for producing voltage pulses which can be calibrated for various voltage amplitudes, durations and cycles. The syringe can be of a variety of sizes and can be selected to inject compositions of the invention at different delivery depths such as to the skin of an organism such as a mammal, or through the skin.

The term “skin” refers to the outer covering of a mammal consisting of epidermal and dermal tissue and appendages such as sweat ducts and hair follicles. Skin can include the hair of a mammal in cases where the mammal has an epidermis which is covered by hair. In mammals which have enough hair to be considered fur or a pelt it is preferable to shave the hair, leaving primarily skin.

The term “organism” as used herein refers to common usage by one of ordinary skill in the art. The organism can include; micro-organisms, such as yeast or bacteria, plants, birds, reptiles, fish or mammals. The organism can be a companion animal or a domestic animal. Preferably the organism is a mammal and is therefore any warm blooded organism. More preferably the mammal is a human.

The term “companion animal” as used herein refers to those animals traditionally treated as “pets” such as for example, dogs, cats, horses, birds, reptiles, mice, rabbits, hamsters, and the like.

The term “domestic animal” as used herein refers to those animals traditionally considered domesticated, where animals such as those considered “companion animals” are included along with animals such as, pigs, chickens, ducks, cows, goats, lambs, and the like.

The term “immune response” as used herein refers to the mammalian natural defense mechanism that can occur when foreign material is internalized. The immune response can be a global immune response involving the immune system components in their entirety. Preferably the immune response results from the protein product encoded by the nucleic acid molecule of the composition. The immune response can be, but is not limited to; antibody production, T-cell proliferation/differentiation, activation of cytotoxic T-lymphocytes, and/or activation of natural killer cells. Preferably the immune response is a humoral immune response. However, as noted above, in other situations the immune response, preferably, is a cytotoxic T-lymphocyte response.

The term “humoral immune response” refers to the production of antibodies in response to internalized foreign material. Preferably the foreign material is the protein product encoded by a nucleic acid molecule of the composition of the invention, wherein the nucleic acid molecule is internalized by injection with a needle free device.

By “prolong the localized bioavailability of a nucleic acid” is meant that a nucleic acid when administered to an organism in a composition comprising such a compound will be available for uptake by cells for a longer period of time than if administered in a composition without such a compound, for example when administered in a formulation such as a saline solution. This increased availability of nucleic acid to cells could occur, for example, due to increased duration of contact between the composition containing the nucleic acid and a cell or due to protection of the nucleic acid from attack by nucleases. The compounds that prolong the localized bioavailability of a nucleic acid are suitable for internal administration.

By “suitable for internal administration” is meant that the compounds are suitable to be administered within the tissue of an organism, for example within a muscle or within a joint space, intradermally or subcutaneously. Other forms of administration which may be utilized are topical, oral, pulmonary, nasal and mucosal; for example, buccal, vaginal or rectal. Properties making a compound suitable for internal administration can include, for example, the absence of a high level of toxicity to the organism as a whole.

By “sustained-release compound” is meant a substance with a viscosity above that of an isotonic saline solution (150 mM NaCl) containing a nucleic acid; for example, DNA in saline at 1 mg/ml has a viscosity of 3.01 mPasec, DNA in saline at 2 mg/ml has a viscosity of 3.26 mPasec, DNA in saline at 3 mg/ml has a viscosity of 5.85 mPasec (Viscosity measurements were performed at 25° C. in a Brookfield DV-III Rheometer with a No. 40 Spindle at 75 rpm for 30 minutes).

By “sustained-release” is meant that nucleic acid is made available for uptake by surrounding tissue or cells in culture for a period of time longer than would be achieved by administration of the nucleic acid in a less viscous medium, for example, a saline solution.

These sustained-release compounds may be prepared as solutions, suspensions, gels, emulsions or microemulsions of a water/oil (w/o), water/oil/water (w/o/w), oil/water (o/w) or oil/water/oil (o/w/o) type. Oil suspensions of lyophilized nucleic acid, such as plasmid DNA may be utilized. Carriers for these oil suspensions include, but are not limited to, sesame oil, cottonseed oil, soybean oil, lecithins, Tweens, Spans and Miglyols.

By “solutions” is meant water soluble polymers and/or surfactants in solution with nucleic acids.

By “suspensions” is meant water insoluble oils containing suspended nucleic acids.

By “gels” is meant high viscosity polymers containing nucleic acids.

By “emulsion” is meant a dispersed system containing at least two immiscible liquid phases. Emulsions usually have dispersed particles in the 0.1 to 100 micron range. They are typically opaque and thermodynamically unstable. Nucleic acids in the water phase can be dispersed in oil to make a w/o emulsion. This w/o emulsion can be dispersed in a separate aqueous phase to yield a w/o/w emulsion. Alternatively, a suitable oil could be dispersed in an aqueous phase to form an o/w emulsion. A “microemulsion” has properties intermediate to micelles and emulsions and is characterized as homogenous, transparent and thermodynamically stable. They form spontaneously when oil, water, surfactant and cosurfactant are mixed together. Typically, the diameter of the dispersed phase is 0.01 to 0.1 microns, usually of the w/o and o/w type.

Some compounds which prolong the bioavailability of a nucleic acid may also interact or associate with the nucleic acid by intermolecular forces and/or valence bonds such as: Van der Waals forces, ion-dipole interactions, ion-induced dipole interactions, hydrogen bonds, or ionic bonds. These interactions may serve the following functions: (1) stereoselectively protect nucleic acids from nucleases by shielding; (2) facilitate the cellular uptake of nucleic acid by “piggyback endocytosis”. Piggyback endocytosis is the cellular uptake of a drug or other molecule complexed to a carrier that may be taken up by endocytosis. C V Uglea and C Dumitriu-Medvichi, Medical Applications of Synthetic Oligomers, In: Polymeric Biomaterials, Severian Dumitriu ed., Marcel Dekker, Inc., 1993, incorporated herein by reference.

To achieve the desired effects set forth it is desirable, but not necessary, that the compounds that prolong the bioavailability of a nucleic acid have amphiphilic properties; that is, have both hydrophilic and hydrophobic regions. The hydrophilic region of the compounds may associate with the largely ionic and hydrophilic regions of the nucleic acid, while the hydrophobic region of the compounds may act to retard diffusion of nucleic acid and to protect nucleic acid from nucleases. Additionally, the hydrophobic region may specifically interact with cell membranes, possibly facilitating endocytosis of the compound and thereby also of nucleic acid associated with the compound. This process may increase the pericellular concentration of nucleic acid. Agents which may have amphiphilic properties and are generally regarded as being pharmaceutically acceptable are the following: polyglutamic acid; poloxamers (Pluronics®), poloxamines (Tetronics®).

The form of the DNA affects the expression efficiency. Therefore, the DNA preferably is at least about 80% supercoiled, more preferably the DNA is at least about 90% supercoiled, and most preferably the DNA is at least about 95% supercoiled. The composition preferably includes an isotonic carbohydrate solution, such as an isotonic carbohydrate solution that consists essentially of about 10% lactose. The compounds which protect the nucleic acid and/or prolong the localized bioavailability of a nucleic acid may achieve one or more of the following effects, due to their physical, chemical or rheological properties: (1) Protect nucleic acid, for example plasmid DNA, from nucleases due to steric, viscosity, or other effects such as shearing; (2) increase the area of contact between nucleic acid, such as plasmid DNA, through extracellular matrices and over cellular membranes, into which the nucleic acid is to be taken up; (3) concentrate nucleic acid, such as plasmid DNA, at cell surfaces due to water exclusion; (4) indirectly facilitate uptake of nucleic acid, such as plasmid DNA, by disrupting cellular membranes due to osmotic, hydrophobic or lytic effects; (5) indirectly facilitate uptake of nucleic acids by allowing diffusion of protected nucleic acid chains through tissue at the administration site; and (6) indirectly facilitate uptake of nucleic acid molecules through pore, holes, openings in the cells formed as a result of the electroporation process.

The compounds which prolong the bioavailability of a nucleic acid may also interact or associate with the nucleic acid by intermolecular forces and/or valence bonds such as: Van der Waals forces, ion-dipole interactions, ion-induced dipole interactions, hydrogen bonds, or ionic bonds. These interactions may serve the following functions: (1) stereoselectively protect nucleic acids from nucleases by shielding; (2) facilitate the cellular uptake of nucleic acid by “piggyback endocytosis”. Piggyback endocytosis is the cellular uptake of a drug or other molecule complexed to a carrier that may be taken up by endocytosis. C V Uglea and C Dumitriu-Medvichi. Medical Applications of Synthetic Oligomers. In: Polymeric Biomaterials. Edited by Severian Dumitriu. Marcel Dekker, Inc. 1993, incorporated herein by reference.

To achieve the desired effects set forth it is desirable, but not necessary, that the compounds which prolong the bioavailability of a nucleic acid have amphipathic properties; that is, have both hydrophilic and hydrophobic regions. The hydrophilic region of the compounds may associate with the largely ionic and hydrophilic regions of the nucleic acid, while the hydrophobic region of the compounds may act to retard diffusion of nucleic acid and to protect nucleic acid from nucleases. Additionally, the hydrophobic region may specifically interact with cell membranes, possibly facilitating endocytosis of the compound and thereby nucleic acid associated with the compound. This process may increase the pericellular concentration of nucleic acid.

Agents which may have amphipathic properties and are generally regarded as being pharmaceutically acceptable are the following: poloxamers (Pluronics®); poloxamines (Tetronics®); methylcelluloses, hydroxypropylcelluloses, hydroxypropylmethylcelluloses; heteropolysaccharides (pectins); ethylene vinyl acetates; polyethylene glycols; polyvinylpyrrolidones; chitosans; polyvinylalcohols; polyvinylacetates; phosphatidylcholines (lecithins); propylene glycol; miglyols; polylactic acid; polyhydroxybutyric acid; xanthan gum. Also, copolymer systems such as polyethylene glycol-polylactic acid (PEG-PLA), polyethylene glycol-polyhydroxybutyric acid (PEG-PHB), polyvinylpyrrolidone-polyvinylalcohol (PVP-PVA), and derivatized copolymers such as copolymers of N-vinyl purine (or pyrimidine) derivatives and N-vinylpyrrolidone.

As used herein the term “poloxamer” means any di- or tri-block copolymer composed of propylene oxide and ethylene oxide. Poloxamers of the Pluronic® type are tri-block copolymers in which the propylene oxide block is sandwiched between two ethylene oxide blocks and has the following general formula and structure:

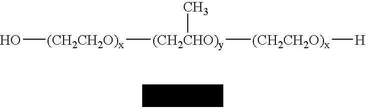

Poloxamers of the reverse Pluronic® type have the following structure:

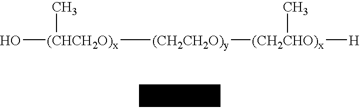

In the nomenclature of poloxamers, the non-proprietary name “poloxamer” is followed by a number, the first two digits of which, when multiplied by 100, equals the average molecular weight (“mw”) of the polyoxypropylene (POP) and the third digit, when multiplied by 10 equals the approximate weight percent of the polyoxyethlyene (POE). Thus, for example, poloxamer 188 would have an average POP mw of about 1800 and an average POE % of 80%. (Lorraine Reeves, Handbook of Biodegradable Polymers, Chapter 12, “The Poloxamers: Their Chemistry and Medical Applications”, in Drug Targeting and Delivery Series, Vol. 7, Harwood Academic Publishers, 1997.)

Calculated according to the poloxamer nomenclature, poloxamer 188 (or F68) would have an average number of POP equal to: 1800÷58 (mw of C₃H₆O)=31 POP units. The total molecular weight of the poloxamer would be: 1800÷(20/100)=9000. The average number of POE would be: (total ˜mw−mw POP)÷44 (mw of C₂H₄O); i.e., (9000−1800)=7200÷44=163. Therefore the formula for poloxamer 188 (or F68) would be:

HO—(C₂H₄O)₈₂—(C₃H₆O)₃₁—(C₂H₄O)₈₂—H

On the other hand, calculated from the formula HO—(C₂H₄O)ₓ—(C₃H₆O)_{y}—(C₂H₄O)ₓ—H, the average molecular weight, and the percentage of POE, the numbers of POE and POP units can be otherwise derived depending on the variable known. Thus, for exampe, if the total mw and % POE is known, the formula is derived as follows:

Ave. # POE are derived as follows: ((total ˜mw−18)×wt % POE)=mw POE÷44=# POE i.e. f/F68; ((8400−18)×80%)=6705.6÷44=152.4(÷2=76)

Average # POP are derived as follows: ((total ˜mw−18)−mw POE)=mw POP÷58 i.e. f/F68; ((8400−18)−6705.6=1676.4÷58=30

Therefore the formula for poloxamer 188, or F68:

HO—(C₂H₄O)₇₆—(C₃H₆O)₃₀—(C₂H₄O)₇₆—H

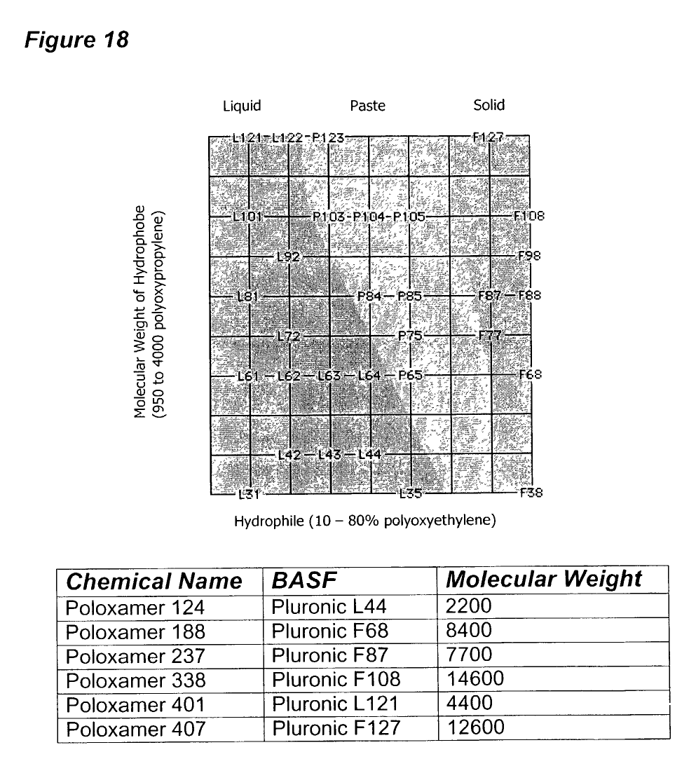

In the BASF nomenclature, a letter describing the physical form of the poloxamer is followed by a first number arbitrarily representing the molecular weight of the POP step-wise up the y axis of the poloxamer grid (FIG. 18) and the second number representing the % POE. Pluronic F68 is the BASF tradename for poloxamer 188. BASF gives 8400 as the average mw for F68 but states an average mw of 8600 for F68 NF grade and gives values of POE=80(×2), and POP=27. Therefore, the POP mw=1566, POE %=81.6% with the resulting formula being HO—(C₂H₄O)₈₀—(C₃H₆O)₂₇—(C₂H₄O)₈₀—H, which would have a resulting mw of 18+7040+1566=8624. (BASF Corporation: Performance Chemicals, Pluronic Grid.)

A description of the method of BASF for manufacturing Pluronic poloxamers demonstrates why any individual poloxamer cannot actually be accurately described by a given molecular formula:

“Synthesizing PLURONIC surfactants is a two-step process:

1. We create a hydrophobe of the desired molecular weight by the controlled addition of propylene oxide to the two hydroxyl groups of propylene glycol.

2. We add ethylene oxide to sandwich the hydrophobe between hydrophilic groups. The hydrophilic groups constitute from 10% to 80% (by weight) of the final molecule.

Since both the ratio and weights of EO and PO vary within this family of surfactants, a PLURONIC Grid was developed to provide a graphic representation of the relationship between copolymer structure, physical form and surfactant characteristics.” http://www.basf.com/static/OpenMarket/Xcelerate/Preview_cid-982931199819 pubid-974236729499 c-Article.html

The following is a table summarizes the average characteristics of some poloxamers:

| | | | Ave. # | Ave. # | | | |
|---|---|---|---|---|---|---|---|
| | | BASF mw | POP | POE | Wt % | MW | Formula^{R} |
| BASF* | Poloxamer | Range | units | units | POE | POP | Formula Derived f/BASF |
| L44NF | Poloxamer 124 | 2200 | 20 | 24.0 | 46.7 ± 1.9 | 1160 | HO—(C₂H₄O)₁₂—(C₃H₆O)₂₀—(C₂H₄O)₁₂—H |
| | | (2090-2360) | | | | | |
| L121 | Poloxamer 401 | 4400^{R} | 67^{R} | 12^{R} | 12^{R} | 3886^{R} | HO—(C₂H₄O)₆—(C₃H₆O)₆₂—(C₂H₄O)₆—H^{R} |
| F68NF | Poloxamer 188 | 8400 | 27 | 160 | 81.8 ± 1.9 | 1800 | HO—(C₂H₄O)₈₀—(C₃H₆O)₂₇—(C₂H₄O)₈₀—H |
| | | (7680-9510) | | | | | |
| F87NF | Poloxamer 237 | 7700 | 37 | 128 | 72.4 ± 1.9 | | HO—(C₂H₄O)₆₄—(C₃H₆O)₁₇—(C₂H₄O)₆₄—H |
| | | (6840-8830) | | | | | |
| F108NF | Poloxamer 338 | 14600 | 44 | 282 | 83.1 ± 1.7 | 3250 | HO—(C₂H₄O)₁₄₁—(C₃H₆O)₄₄—(C₂H₄O)₁₄₁—H |
| | | (12700-17400) | | | | | |
| F127NF | Poloxamer 407 | 12600 | 56 | 202 | 73.2 ± 1.7 | 11716 | HO-13 (C₂H₄O)₁₀₁ —(C₃H₆O)₅₆ —(C₂H₄O)₁₀₁—H |
| | | (9840-14600) | | | | | |

*Values taken from BASF NF Grade Pluronic Polymers Technical Bulletin Sep. 17, 2001 unless indicated
^{R}Values taken from Lorraine Reeves, Handbook of Biodegradable Polymers, Chapter 12, in Drug Targeting and Delivery Series, Vol. 7, Harwood Academic Publishers, 1997.

Another way of describing the characteristics of various poloxamers is by reference to the Pluronic Surfactant Grid provided in FIG. 18. The PLURONIC® Surfactant Grid is a graphic presentation of the PLURONIC surfactant series. Plotting molecular weight ranges of the hydrophobe (propylene oxide) against the weight-percent of the hydrophile (ethylene oxide) present in each molecule allows property trends of the product structure to be analyzed on the Grid. The Grid also clarifies the use of the letter-number combinations to identify the various products of the PLURONIC series. The alphabetical designation explains the physical form of the product: ‘L’ for liquids, ‘P’ for pastes, ‘F’ for solid forms. The first digit (two digits in a three-digit number) in the numerical designation, multiplied by 300, indicates the approximate molecular weight of the hydrophobe (vertical axis at the left of the Grid). The last digit, when multiplied by 10, indicates the approximate ethylene oxide content in the molecule, read from the horizontal axis. (BASF Corporation: Performance Chemicals, Nomenclature and the PLURONIC® Surfactant Grid).

As used herein, the term “poloxamine” refers to poly(oxyethylene)-poly(oxypropylene) (POE-POP) block copolymers where a POE-POP unit is linked to another POE-POP unit by an amine and having the general structure (POEₙ-POPₘ)₂—N—C₂H₄—N−(POPₘ-POEₙ)₂. TETRONIC® and TETRONIC® nonionic surfactants produced by BASF are exemplary poloxamines. TETRONIC® 904 is supplied as a liquid having an average molecular weight of 6,700 Da. TETRONIC® 908 is supplied as a solid having an average molecular weight of 25,000 Da. TETRONIC® 1107 is supplied as a solid having an average molecular weight of 15,000 Da. TETRONIC® 90R4 is supplied as a liquid having an average molecular weight of 7,240 Da.

The delivery of compositions of the invention by the use of pulse voltage delivery device represents a novel approach to gene delivery. The invention provides the advantage of allowing the uptake of formulated nucleic acid molecules (i.e., nucleic acid molecules in the compositions of the invention) by specifically targeted cells and cell lines, as well as uptake by multiple cell lines as desired. Injecting formulated nucleic acid molecules by pulse voltage delivery methods results in the formulated nucleic acid molecules gaining access to the cellular interior more directly through the destabilization of the cell wall and/or by the formation of pores as a result of the electroporetic process. Furthermore, in certain instances multiple cell lines can be targeted, thus allowing contact to many more cell types than in conventional needle injection. Thus, the present invention provides an enhanced delivery of nucleic acid molecules and also provides a more efficient gene delivery system which can be used to generate an immune response, modulate aspects of the cell cycle or cell physiology, or provide a method to achieve other gene delivery related therapeutic methods such as anti-tumor therapy.

Polymeric and Non-Polymeric Formulations for Plasmid Delivery to Muscle

The present invention provides polymeric and non-polymeric formulations which address problems associated with injection of nucleic acids suspended in saline. Unformulated (naked nucleic acid molecules) plasmids suspended in saline have poor bioavailability in muscle due to rapid degradation of plasmid by extracellular nucleases. One possible approach to overcome the poor bioavailability is to protect plasmid from rapid nuclease degradation by, for example, condensing the plasmid with commonly used cationic complexing agents. However, due to the physiology of the muscle, the use of rigid condensed particles containing plasmid for efficient transfection of a larger number of muscle cells has not been successful to date. Cationic lipid and polylysine plasmid complexes do not cross the external lamina to gain access to the caveolae and T tubules [Wolff, J. A., et al., 1992, J. Cell. Sci. 103:1249-1259].

Thus, the invention increases the bioavailability of plasmid in muscle by: protecting plasmid from rapid extracellular nuclease degradation, dispersing and retaining intact plasmid in the muscle and/or tumor, and facilitating the uptake of plasmid by muscle and/or tumor cells. A specific method of accomplishing this, which preferably is used in conjunction with pulse voltage delivery, is the use of protective, interactive, non-condensing systems (PINC).

Exemplary Polymeric Sustained Release Systems

Due to the rapid rate at which plasmid formulated in saline is degraded and/or removed from the site of injection, one strategy is to develop systems with increased viscosity to retain plasmid at the site of injection. Further, since the uptake of plasmid appeared to be a saturable process, maintaining a high concentration of plasmid in muscle for a prolonged period of time may enhance plasmid bioavailability in muscle [March, K. L., et al., 1995, Hum. Gene Ther. 6:41-53; Mathiowitz, E., et al., (Sep. 21, 1995), Polymeric gene delivery systems WO 95/24929].

In an alternative embodiment a thermoreversible gel may be used. After IM administration, plasmid DNA is maintained within the muscle by using a thermo-reversible gel formulation. The use of compounds that are aqueous at ambient temperature, yet are gels at body temperatures (e.g. 37° C. for humans) are used to ease the formulation and administration of the DNA, yet transition to and maintain the gel state for increased bio-availability at temperatures encountered in vivo.

Such formulations (thermo-reversible gels) are prepared by adjusting the concentrations of polymers in aqueous solutions so that the vector delivery system will be liquid at room temperature or below and will be in the form of a gel in situ in the muscle at physiologic temperatures. Poloxamers (PLURONIC® L44, F68, F87, F108, L121, F127) or poloxamines concentrations may be adjusted according to the formulation depending upon the route of administration (i.e., topical, i.m,) for nucleic acid or nucleic acid complexes.

These adjustments, for example, may be found in U.S. Pat. No. 5,292,516, incorporated by reference herein. By “thermo-reversible gel” is meant a gel which is substantially liquid at temperatures below about 30° C. but forms a gel at temperatures above about 30° C. Administration of the thermo-reversible gel by, for example, injection is thereby facilitated if the gel is cooled so that it is in a substantially liquid state when injected. However, upon contact with the tissue of an organism which is at a temperature of above about 30° C. the viscosity of the thermo-reversible gel increases, thereby increasing the localized bioavailability of a nucleic acid formulated with the thermo-reversible gel.

Protective, Interactive Non-Condensing (PINC) Systems

The information described herein can be used to design novel co-polymers that will also have enhanced interaction with plasmids. It is expected that there is “an interactive window of opportunity” whereby enhanced binding affinity of the PINC systems will result in a further enhancement of gene expression after their intramuscular injection due to more extensive protection of plasmids from nuclease degradation. It is expected that there will be an optimal interaction beyond which either condensation of plasmids will occur or “triplex” type formation, either of which can result in decreased bioavailability in muscle and consequently reduced gene expression.

As indicated above, the PINC compunds are generally amphiphilic compounds having both a hydrophobic portion and a hydrophilic portion. In many cases the hydrophilic portion is provided by a polar group. It is recognized in the art that such polar groups can be provided by groups such as, but not limited to, pyrrolidone, alcohol, acetate, amine or heterocyclic groups such as those shown on pp. 2-73 and 2-74 of CRC Handbook of Chemistry and Physics (72nd Edition), David R. Lide, editor, including pyrroles, pyrazoles, imidazoles, triazoles, dithiols, oxazoles, (iso)thiazoles, oxadiazoles, oxatriazoles, diaoxazoles, oxathioles, pyrones, dioxins, pyridines, pyridazines, pyrimidines, pyrazines, piperazines, (iso)oxazines, indoles, indazoles, carpazoles, and purines and derivatives of these groups, hereby incorporated by reference.

The compounds also contain hydrophobic groups which, in the case of a polymer, are typically contained in the backbone of the molecule, but which may also be part of a non-polymeric molecule. Examples of such hydrophobic backbone groups include, but are not limited to, vinyls, ethyls, acrylates, acrylamides, esters, celluloses, amides, hydrides, ethers, carbonates, phosphazenes, sulfones, propylenes, and derivatives of these groups. The polarity characteristics of various groups are quite well known to those skilled in the art as illustrated, for example, by discussions of polarity in any introductory organic chemistry textbook.

The ability of such molecules to interact with nucleic acids is also understood by those skilled in the art, and can be predicted by the use of computer programs which model such intermolecular interactions. Alternatively or in addition to such modeling, effective compounds can readily be identified using one or more of such tests as 1) determination of inhibition of the rate of nuclease digestion, 2) alteration of the zeta potential of the DNA, which indicates coating of DNA, 3) or inhibition of the ability of intercalating agents, such as ethidium bromide to intercalate with DNA.

Targeted Delivery of Nucleic Acid/PINC/Targeting Ligand Complex

In addition to the nucleic acid/PINC complexes described above for delivery and expression of nucleic acid sequences, in particular embodiments it is also useful to provide a targeting ligand in order to preferentially obtain expression in particular tissues, cells, or cellular regions or compartments.

Such a targeted PINC complex includes a PINC system (monomeric or polymeric PINC compound) complexed to plasmid (or other nucleic acid molecule). The PINC system is covalently or non-covalently attached to (bound to) a targeting ligand (TL) that binds to receptors having an affinity for the ligand. Such receptors may be on the surface or within compartments of a cell. Such targeting provides enhanced uptake or intracellular trafficking of the nucleic acid.

The targeting ligand may include, but is not limited to, galactosyl residues, fucosal residues, mannosyl residues, carnitine derivatives, monoclonal antibodies, polygonal antibodies, peptide ligands, and DNA-binding proteins. Examples of cells that may usefully be targeted include, but are not limited to, antigen-presenting cells, hepatocytes, myocytes, epithelial cells, endothelial cells, and cancer cells.

Formation of such a targeted complex is illustrated by the following example of covalently attached targeting ligand (TL) to PINC system:

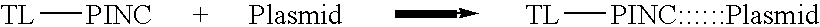

Formation of such a targeted complex is also illustrated by the following example of non-covalently attached targeting ligand (TL) to PINC system

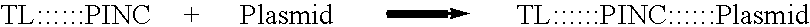

or alternatively,

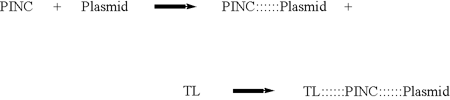

In these examples, ::::::: is non-covalent interaction such as ionic, hydrogen-bonding, Van der Waals interaction, hydrophobic interaction, or combinations of such interactions.

A targeting method for cytotoxic agents is described in Subramanian et al., International Application No. PCT/US96/08852, International Publication No. WO 96/39124, hereby incorporated by reference. This application describes the use of polymer affinity systems for targeting cytotoxic materials using a two-step targeting method involving zip polymers, i.e., pairs of interacting polymers. An antibody attached to one of the interacting polymers binds to a cellular target. That polymer then acts as a target for a second polymer attached to a cytotoxic agent. As referenced in Subramanian et al., other two-step (or multi-step) systems for delivery of toxic agents are also described.

In another aspect, nucleic acid coding sequences can be delivered and expressed using a two-step targeting approach involving a non-natural target for a PINC system or PINC-targeting ligand complex. Thus, for example, a PINC-plasmid complex can target a binding pair member which is itself attached to a ligand which binds to a cellular target (e.g., a MAB). Binding pairs for certain of the compounds identified herein as PINC compounds are identified in Subramanian et al. Alternatively, the PINC can be complexed to a tareting ligand, such as an antibody. That antibody can be targeted to a non-natural target that binds to, for example a second antibody.

Preparation of Formulations

Formulations of nucleic acid molecules can be prepared as disclosed herein. Substitute polymers are selected as determined by application. Generally, a weight/volume ratio is used as exemplified in both of the provided examples.

Delivery and expression of nucleic acids in many formulations, such as in saline, is limited due to degradation of the nucleic acids by cellular components of organisms, such as for instance nucleases. Thus, protection of the nucleic acids when delivered in vivo can greatly enhance the resulting expression, and thereby enhance a desired pharmacological or therapeutic effect. It was found that certain types of compounds that interact with a nucleic acid (e.g., DNA) in solution but do not condense the nucleic acid provide in vivo protection to the nucleic acid, and correspondingly enhance the expression of an encoded gene product. Some of these compounds have been discussed in U.S. Pat. No. 08/484,777, filed Jun. 7, 1998, International Patent Application No. PCT/US96/05679 filed Apr. 23, 1996 and U.S. Patent Application Serial No. 60/045,295, filed May 2, 1997 all of which are incorporated herein by reference in their entirety including any drawings.

The use of delivery systems designed to interact with plasmids and protect plasmids from rapid extracellular nuclease degradation are described in, Mumper, R. J., et al., 1996, Pharm. Res. 13:701-709; Mumper, R. J., et al., 1998, J. Controll. Release 52:191-203; Anwer K et al. 1998, Human Gene Therapy, 9:659-670; and Alila H et al., 1997, Human Gene Therapy 8:1785-1795. A characteristic of the PINC systems is that they are non-condensing systems that allow the plasmid to maintain flexibility and diffuse freely throughout the muscle while being protected from nuclease degradation. While the PINC systems are primarily discussed below, it will be understood that cationic lipid based systems and systems utilizing both PINCS and cationic lipids are also within the scope of the present invention.

A common structural component of the PINC systems is that they are amphiphilic molecules, having both a hydrophilic and a hydrophobic portion. The hydrophilic portion of the PINC is meant to interact with plasmids by hydrogen bonding (via hydrogen bond acceptor or donor groups), Van der Waals interactions, or/and by ionic interactions. For example, PVP and N-methyl-2-pyrrolidone (NM2P) are hydrogen bond acceptors while PVA and Propylene Glycol (PG) are hydrogen bond donors.

All four molecules have been reported to form complexes with various (poly)anionic molecules [Buhler V., BASF Aktiengescellschaft Feinchemie, Ludwigshafen, pp 39-42; Galaev Y, et al., J. Chrom. A. 684:45-54 (1994); Tarantino R, et al. J. Pharm. Sci. 83:1213-1216 (1994); Zia, H., et al., Pharm. Res. 8:502-504 (1991)]. The hydrophobic portion of the PINC systems is designed to result in a coating on the plasmid rendering its surface more hydrophobic. Kabanov et al. have described previously the use of cationic polyvinyl derivatives for plasmid condensation designed to increase plasmid hydrophobicity, protect plasmid from nuclease degradation, and increase its affinity for biological membranes [Kabanov, A. V., and Kabanov, V. A., 1995, Bioconj. Chem. 6:7-20; Kabanov, A. V., et al., 1991, Biopolymers 31:1437-1443; Yaroslavov, A. A., et al., 1996, FEBS Letters 384:177-180].

A substantial protective effect is observed; up to at least a one log enhancement of gene expression in rat muscle over plasmid formulated in saline has been demonstrated with these exemplary non-ionic PINC systems disclosed herein. We have also found that the expression of reporter genes in muscle using plasmids complexed with the PINC systems was more reproducible than when the plasmid was formulated in saline. For example, the coefficient of variation for reporter gene expression in muscle using plasmid formulated in saline was 96±35% (n=20 studies; 8-12 muscles/study) whereas with coefficient of variation with plasmids complexed with PINC systems was 40±19% (n=30 studies; 8-12 muscles/study). The high coefficient of variation for reporter gene expression with plasmid formulated in saline has been described previously [Davis, H. L., et al., 1993, Hum. Gene Ther. 4:151-9]. In addition, in contrast with the results for DNA:saline, there was no significant difference in gene expression in muscle when plasmid with different topologies were complexed with polyvinyl pyrrolidone (PVP). This suggests that PVP is able to protect all forms of the plasmid from rapid nuclease degradation.

Administration

Administration as used herein refers to the route of introduction of a plasmid or carrier of DNA into the body. Administration can be directly to a target tissue or by targeted delivery to the target tissue after systemic administration. In particular, the present invention can be used for treating conditions by administration of the formulation to the body in order to establish controlled expression of any specific nucleic acid sequence within tissues at certain levels that are useful for gene therapy.

The preferred means for administration of vector (plasmid) and use of formulations for delivery are described above. The preferred embodiments are by pulse voltage delivery to cells in combination with needle or needle free injection, or by direct applied pulse voltage wherein the electroporation device's electrodes are pressed directly against the targeted tissue or cells, such as for example epidermal cells, and the vector is applied topically before or after pulse application and delivered through and or to the cells.

The route of administration of any selected vector construct will depend on the particular use for the expression vectors. In general, a specific formulation for each vector construct used will focus on vector delivery with regard to the particular targeted tissue, the pulse voltage delivery parameters, followed by demonstration of efficacy. Delivery studies will include uptake assays to evaluate cellular uptake of the vectors and expression of the DNA of choice. Such assays will also determine the localization of the target DNA after uptake, and establishing the requirements for maintenance of steady-state concentrations of expressed protein. Efficacy and cytotoxicity can then be tested. Toxicity will not only include cell viability but also cell function.

Muscle cells have the unique ability to take up DNA from the extracellular space after simple injection of DNA particles as a solution, suspension, or colloid into the muscle. Expression of DNA by this method can be sustained for several months.

The chosen method of delivery should result in expression of the gene product encoded within the nucleic acid cassette at levels that exert an appropriate biological effect. The rate of expression will depend upon the disease, the pharmacokinetics of the vector and gene product, and the route of administration, but should be in the range 0.001-100 mg/kg of body weight/day, and preferably 0.01-10 mg/kg of body weight/day. This level is readily determinable by standard methods. It could be more or less depending on the optimal dosing. The duration of treatment will extend through the course of the disease symptoms, possibly continuously. The number of doses will depend upon the disease, delivery vehicle, and efficacy data from clinical trials.

DNA Injection Variables

The level of gene delivery and expression or the intensity of an immune response achieved with the present invention can be optimized by altering the following variables. The variables are: the formulation (composition, plasmid topology), the technique and protocol for injection (area of injection, duration and amplitude of voltage, electrode gap, number of pulses emitted, type of needle arrangement, pre-injection-pulsed or post-injection-pulsed cells, state of muscle, state of the tumor), and, the pretreatment of the muscle with myotoxic agents. An immune response can be measured by, but is not limited to, the amount of antibodies produced for a protein encoded and expressed by the injected nucleic acid molecule.

Other injection variables that can be used to significantly affect the levels of proteins, antibodies and/or cytotoxic T-lymphocytes produced in response to the protein encoded by the formulated nucleic acid molecule provided by the pulse voltage injection method of the present invention are the state of the muscle being injected and injection technique. Examples of the variables include muscle stimulation, muscle contraction, muscle massage, delivery angle, and apparatus manipulation. Massaging the muscle may force plasmid out of the muscle either directly or via lymphatic drainage. By altering the depth of penetration and/or the angle at which the pulse voltage device is placed in relation to muscle fibers the present invention improves the plasmid distribution throughout the injection area that subsequently increases the antibody response to the protein that is encoded and expressed by the plasmid.

Nucleic Acid Based Therapy

The present invention can be used to deliver nucleic acid vaccines in a more efficient manner than is conventionally done at the present time. Nucleic acid vaccines, or the use of plasmid encoding antigens or therapeutic molecules such as Human Growth Hormone, has become an area of intensive research and development in the last half decade. Comprehensive reviews on nucleic acid based vaccines have been published [M. A. Liu, et al.(Eds.), 1995, DNA Vaccines: A new era in vaccinology, Vol. 772, Ann. NY. Acad. Sci., New York; Kumar, V., and Sercarz, E., 1996, Nat. Med. 2:857-859; Ulmer, J. B., et al., (Eds.) Current Opinion in Immunology; 8:531-536. Vol. 772, Ann. NY. Acad. Sci., New York]. Protective immunity in an animal model using plasmid encoding a viral protein was first observed in 1993 by Ulmer et al. [Ulmer, J. B., et al., 1993, Science 259:1745-1749]. Since then, several studies have demonstrated protective immunity for several disease targets and human clinical trials have been started.

Many disease targets have been investigated. Examples include antigens of Borrelia burgdorferi, the tick-borne infectious agent for Lyme disease (Luke et al., J. Infect. Dis. 175:91-97, 1997), human immunodeficiency virus-1, (Letvin et al., Proc. Nat. Acad. Sci. USA 94:9378-9383, 1997), B cell lymphoma (Syrengelas et al., Nature Medicine. 2:1038-41, 1996), Herpes simplex virus (Bourne et al., J. Infectious dis. 173:800-807, 1996), hepatitis C virus (Tedeschi et al., Hepatology 25:459-462, 1997), rabies virus (Xiang et al., virology, 209:569-579, 1995), Mycobacterium tuberculosis (Lowrie in Genetic Vaccines and Immunotherapeutic Strategies CA Thibeault, ed. Intl Bus Comm, Inc., southborough, MA 01772 pp. 87-122, 1996), and Plasmodium falciparum (Hoffman et al., Vaccine 15:842-845, 1997). Additionally, nucleic acid based treatment for reducing tumor-cell immunogenicity, growth, and proliferation is indicative of gene therapy for diseases such as tumorigenic brain cancer (Fakhrai et al., Proc. Natl. Acad. Sci., 93:2909-2914, 1996).

An important goal of gene therapy is to affect the uptake of nucleic acid by cells, thereby causing an immune response to the protein encoded by the injected nucleic acid. Uptake of nucleic acid by cells is dependent on a number of factors, one of which is the length of time during which a nucleic acid is in proximity to a cellular surface. The present invention provides formulations that increase the length of time during which a nucleic acid is in proximity to a cellular surface, and can penetrate the cell resulting in delivery of nucleic acid molecules into the cell.

Nucleic acid based vaccines are an attractive alternative vaccination strategy to subunit vaccines, purified viral protein vaccines, or viral vector vaccines. Each of the traditional approaches has limitations that are overcome if the antigen(s) is expressed directly in cells of the body. Furthermore, these traditional vaccines are only protective in a strain-specific fashion. Thus, it is very difficult, and even impossible using traditional vaccine approaches to obtain long lasting immunity to viruses that have several sera types or viruses that are prone to mutation.

Nucleic acid based vaccines offer the potential to produce long lasting immunity against viral epitopes that are highly conserved, such as with the nucleoprotein of viruses. Injecting plasmids encoding specific proteins by the present invention results in increased immune responses, as measured by antibody production. Thus, the present invention includes new methods of providing nucleic acid vaccines by delivering a formulated nucleic acid molecule with a pulse voltage device as described herein.

The efficacy of nucleic acid vaccines is enhanced by one of at least three methods: (1) the use of delivery systems to increase the stability and distribution of plasmid within the muscle, (2) by the expression (or delivery) of molecules to stimulate antigen presentation/transfer, or (3) by the use of adjuvants that may modulate the immune response.

Diseases and Conditions for Intramuscular Plasmid Delivery

The present invention described herein can be utilized for the delivery and expression of many different coding sequences. In particular, the demonstrated effectiveness for the PINC systems (PCT Application No. PCT/US96/05679) for delivery to muscle indicate that such formulations are effective for delivery of a large variety of coding sequences to muscle by pulse voltage injection. As transforming muscle and other cells has been shown to be effective, in an additional aspect of the invention tumor cells are also targeted for pulse voltage injection. Hence, the present invention provides methods for treating cancerous conditions associated with the formation of tumors or aggregated cell colonies such as those found in conditions such as skin cancer and the like. Specific suggestions for delivery of coding sequences to muscle cells with the pulse voltage device of the present invention include those summarized in Table 1 below.

TABLE 1
APPLICATIONS FOR PLASMID-BASED GENE THERAPY BY INTRAMUSCULAR
INJECTION

| | |
|---|---|
| Muscle and nerve disorders | |
| Duchenne's muscular dystrophy | Acsadi 1991 [5], Karpati 1993 [6], Miller 1995 [7] |
| Myotrophic disorders (IGF-I) | Coleman 1997 [8], Alila 1997 [9] |
| Neurotrophic disorders (IGF-I) | Alila 1997 [9], Rabinovsky 1997 [10] |
| Secretion of expressed protein | |
| into the systemic circulation | |
| Hemophilias A and B | Anwer 1996 [11], Kuwahara-Rundell 1994 [12], Miller |
| | 1994 [13] |
| ERYTHROPOIETIN-RESPONSIVE1996 | Tripathy [14] |
| Pituitary dwarfism | AnWer 1996 [11], DAHLER 1994 [15] |
| α1-Antitrypsin deficiency | Levy 1996 [16] |
| Autoimmune and Inflammatory | Raz 1993 [17] |
| diseases | |
| Hypercholesterolema | Fazio 1994 [18] |
| Hypotension | Ma 1995 [19] |
| Hypertension | Xiong 1995 [20] |
| Nucleic acid vaccines | |
| Herpes Simplex Virus | Manickan 1995 [21], Ghiasi 1995 [22], McClements |
| | 1996 [23], Kriesel 1996 [24] |
| Hepatitis B Virus | Davis 1993 [25], Davis 1994 [26], Davis 1996 [27] |
| Influenza Virus | Donnelly 1995 [28], Ulmer 1993 [29], Ulmer 1994 [30] |
| Tuberculosis | Lowrie 1994 [31], Tascon, 1996 [32] |
| Human Immunodeficiency Virus | Shiver 1995 [33], Coney 1994 [34], Wang 1993 [35] |
| Cancer | Raz 1993 [17], Russell 1994 [36] |
| Maleria | Hoffman 1995 [37], Sedegah 1994 [38] |
| Hepatitis C virus | Major 1995 [39], Lagging 1995 [40] |
| Flavivirus | Phillpotts 1996 [41] |
| Cytomegalovirus | Pande 1995 [42] |
| Salmonella typhi | Lopez-Macias 1995 [43] |
| Mycoplasma pulmonis | Lai 1995 [44] |
| Rabies virus | Xiang 1995 [45] |

REFERENCES ARE NUMBERED AS THEY ARE CITED IN U.S. APPLICATION No. PCT/US96/05679, WHICH HAS BEEN INCORPORATED BY REFERENCE IN ITS ENTIRETY.

The condition or disease preferably is a cancer, such as epithelial glandular cancer, including adenoma and adenocarcinoma; squamous and transitional cancer, including polyp, papilloma, squamous cell and transitional cell carcinoma; connective tissue cancer, including tissue type positive, sarcoma and other (oma's); hematopoietic and lymphoreticular cancer, including lymphoma, leukemia and Hodgkin's disease; neural tissue cancer, including neuroma, sarcoma, neurofibroma and blastoma; mixed tissues of origin cancer, including teratoma and teratocarcinoma. Other cancerous conditions that are applicable to treatment include cancer of any of the following: adrenal gland, anus, bile duct, bladder, brain tumors: adult, breast, cancer of an unknown primary site, carcinoids of the gastrointestinal tract, cervix, childhood cancers, colon and rectum, esophagus, gall bladder, head and neck, islet cell and other pancreatic carcinomas, Kaposi's sarcoma, kidney, leukemia, liver, lung: non-small cell, lung: small cell, lymphoma: AIDS-associated, lymphoma: Hodgkin's disease, Lymphomas: non-Hodgkin's disease, melanoma, mesothelioma, metastatic cancer, multiple myeloma, ovary, ovarian germ cell tumors, pancreas, parathyroid, penis, pituitary, prostate, sarcomas of bone and soft tissue, skin, small intestine, stomach, testis, thymus, thyroid, trophoblastic disease, uterus: endometrial carcinoma, uterus: uterine sarcomas, vagina, or vulva. The composition preferably is administered by pulsed voltage delivery and may require, as needed, exposure of the tissue to be treated by surgical means as determined by a certified professional.

### EXAMPLE I

### Materials and Methods

The following examples are offered by way of illustration and are not intended to limit the scope of the invention in any manner. One of ordinary skill in the art would recognize that the various molecules and/or amounts disclosed in the examples could be adjusted or substituted. It would also be recognized that the delivery targets and/or amounts delivered in the examples could be adjusted or substituted by selecting different muscles for injection, injection into tumors or nodes, or increasing or decreasing the duration of pulse time or alternating the pulse application from pre-injection to post-injection.

Materials

USP/NF grade Pluronic® F68 (Poloxamer 188), Pluronic® F87 (Poloxamer 237), Pluronic® L121 (poloxamer 401), Pluronic® F108 (Poloxamer 338), Pluronic® F127 (Poloxamer 407), Pluronic® L44 (Poloxamer 124), and poloxamines (Tetronics®) were obtained from Spectrum Quality Products, Inc., (New Brunswick, N.J.) and the BASF Corporation (Mount Olive, N.J.). Plasmids containing a CMV promoter and luciferase or GFP reporters were manufactured and purified at Valentis, Inc.

Preparation of Formulations

Formulations were made by aliquoting appropriate volumes of sterile stock solutions of water, plasmid, polymer, and 5M NaCl to obtain a final plasmid in an isotonic polymer solution. The total plasmid concentration of all formulations was measured by UV absorption at 260 nm. The osmotic pressure of selected formulations was measured using a Fiske One-Ten Micro-Sample Osmometer (Fiske Associates; Norwood, Mass.). The percentage of supercoiled plasmid was measured using 1% agarose gel electrophoresis followed by fluorimaging.

Animal Injections

Female CD-1 mice (50-60 g) were purchased from Charles River, Inc. and housed in Laboratory Animal Resources vivarium at Valentis. The animals were anesthetized by intraperitoneal administration of a mixture of ketamine (42.8 mg/ml), xylazine (8.6 mg/ml), and acepromazine (1.4 mg/ml) at a dose of 1.8-2.0 mg/kg. Hind limbs were shaved and scrubbed with betadine followed by 70% ethanol. 10 μL of the formulation was injected with 10 μg of formulated plasmid using a 0.3-ml insulin syringe with a 28-gauge, 0.5 needle (Becton Dickinson, Granklin Lake, N.J.). Seven days after formulation injection, the animals were sacrificed by CO₂ asphyxiation and the tibialis antrior muscles were harvested, quickly immersed in liquid nitrogen, and lyophilized overnight. The dried muscles were used or stored at −80° C. for further determination of report gene activity.

Luciferase Activity and Total Protein Assays

The lyophilized muscles were homogenized using mini bead-beater (biospec Products, Bartlesville, Okla.) with silica beads for 1-2 minutes. 0.5 mL of luciferase cell lysis buffer (Promega, Madison, Ill.) was added to the powdered muscle and the samples were homogenized for another 2-3 minutes. The suspension was centrifuged at 13,000 rpm for 15 minutes. A 20 μL sample of the supernatant (diluted appropriately with 0.5× lysis buffer) was added into 96 microplate. The luciferase activity was assayed by injecting 100 μL reconstituted luciferase assay solution (Promega, Madison, Ill.) using a luminometer (Microlumat LB 96p, Wallac Inc., Gaithersburg, Md.) and relative light units were recorded. The total protein was determined with the BCA protein assay kit (Pierce, Rockford, Ill.).

Histologic Analysis

For the histology assay of the gene expression in the muscle, formulated GFP report gene was injected in the tibialis anterior muscles. Five days after injection, the muscle was harvested and placed in 10% Neutral Buffered Formalin for 6 hours at room temperature. The tissue was processed in paraffin and 5 μm sections were cut and dried for one hours in a 60° C. oven. The samples were subsequently cleared in xylene and rehydrated in PBS. Following three washes with PBS, the samples were covered with cover slip using Vectashield mounting media (Vector Laboratories, Burlingame, Calif.).

Stability Test for Plasmid in the Formulation

For the analysis of pDNA stability in the formulation, 50 ng of formulated pDNA with 5 μL of tracking dye was loaded into 1% agarose gel in 1% tris-acetate-EDTA (TAE) buffer and run the gel at 100 volts for 1-2 hours. The gel was then stained with SYBR Green II (Molecular Probes, Inc.) for 20 minutes. The stained gel was washed with water and % of supercoiled and open circled DNA was determined using a FluorImager (Molecular Dynamics Co., Sunnyvale, Calif.).

CTL Assay Protocol:

To set up spleenocyte stimulation, aseptically harvest up to 3 spleens per group and place in sterile media. Dissociate tissue and allow cells to pass through a 70 micrometer cell strainer. Wash cells thoroughly and lyse RBC's. Resuspend cells in 5 mls complete media/spleen (i.e. for 3 spleens, resuspend in 15 mls). Resuspend at a concentration of 10⁸ cells/ml in complete media. For these effector cells, add 4 mls of a 10× stock of mIL-2 and peptide (used for immunization; 100U/ml mIL-2 and 10 micrograms/ml peptide) and 10⁸ cells/ml in 36 mls of media to a T75 flask. Each flask should contain 40 mls with approximately 3×10⁹ total cells. Place flask upright in a 37° C./5% CO₂ incubator for 5 days.

After 5 days, resuspend target cells at 2.5×10⁶ cells/ml and add 150 microCi of Chromium-51 Sulfate. Make 2 tubes. To one tube, add 25 μg/ml of peptide. Incubate tubes at 37° C. for 2 hours. Prepare effector cells by harvesting the T75 flasks and after sufficient washing (at least 3 times) resuspend at 10⁷ cells/ml. After incubating target cells with Chromium-51, wash unbound radioactivity and resuspend at 5×10⁴ cells/ml. To a round bottom 96 well plate, add 100 microliters/well of effectors at concentrations of 100:1, 50:1, 25:1 and 12.5:1. Add 100 microliters/well of Cr⁵-target cells to all wells including wells containing no effectors. To one-half of the target only wells, add 100 μl of 1% Triton X-100. Incubate plates at 37° C. for 6 hours. Harvest plates and count using a WallacL470 Wizard gamma counter.

Elisa Protocol:

Coat high affinity assay plate with antigen diluted in PBS (50 microliters/well). Place at 4° C. overnight. After allowing plate(s) to come to room temperature, block all wells with 200 microliters/well of 4% BSA/4% NGS solution made in 1×PBS/Tween20 for 1 hr at 37° C. Add serum samples (50 microliters/well at a starting dilution of 1:100 in 4% BSA/4% NGS/PBS/Tween20, in duplicate) and incubate for 1-2 hours at 37° C. Wash plate(s) with PBS/Tween 20 and add 50 microliters/well of HRP-conjugated secondary, diluted in 1% BSA, and incubate at 37° C. for 1 hour. Wash plate(s) with PBS/Tween 20 and add 100 microliters/well of TMB Soluble reagent. Incubate at room temperature for 10 minutes and stop the reaction by adding 50 microliters/well of 0.2M H₂SO₄. Read plate(s) at 450 nm.

Cytokine Release Protocol:

Dissociate spleens and allow cells to pass through a 70 micrometer cell strainer. Wash cells thoroughly and lyse RBC's. Resuspend splenocytes at 5×10⁶ cells/ml. Using a flat bottom 96 well plate, titrate the antigen in quadruplicate starting at 20 micrograms/ml (100 microliters/well). Add 100 microliters/well of the splenocytes to each well and incubate for 60 hours in at 37° C. (final starting concentration of the antigen will now be at 10 micrograms/ml). Collect supernatants from each well (quadruplicates can be pooled) and test using a cytokine ELISA kit (IFN-γ, IL-5 and/or IL-10; each can be obtained from Pharmingen).

Plasmids

Plasmids pAP1166 (SEAP), pGF9910 (GFP) and pCL0888 (luciferase) containing a CMV enhancer-promoter and either a human placental secreted alkaline phosphatase reporter gene (SEAP) (pAP1166), Green Fluorescent Protein (pGF9910), or luciferase (pCL0888) were manufactured and purified at Valentis, Inc. The Valentis backbone includes a 107 bp 5′ UTRm (UT12), a 117 bp synthetic intron (ivs8), a kanamycin resistance gene and a PUC12 backbone.

Experimental Animals

Male CD-1 mice (29-31 g) (Charles Rivers Laboratories) and female C57BL/6 mice (7-8 weeks) were acclimatized for a 3-7 day period in a 12 hour light-dark cycle at 23° C./40% RH in accordance with state and federal guidelines. Animals were anesthetized IP with a combination anesthesia (Ketamine 74 mg/mL, Xylazine 3.7 mg/mL and Acepromazine 0.73 mg/mL) at a dose of 1.8-2.0 mL/kg (mice).

Device and Dosing Regimens

Plasmid formulated at the required dose was administered in rodents by longitudinal injection in both tibialis cranialis or in both gastrocnemius muscles (bilateral administration). When electroporating, by holding the entire lower leg between the caliper electrodes good “electrotransfection” could be obtained. Two minutes after injection, an electric field was applied in the form of 2 square wave pulses (one per second) of 25 ms each and 375 V/cm delivered by an Electro Square Porator (T820, BTX, San Diego, Calif.). The clamp electrodes consist of 2 stainless steel parallel plate calipers (1.5 cm²) that are placed in contact with the skin so that the leg is held in a semi-extended position throughout pulse administration. The separation distance of the electrodes is described.

Serum Assays

Blood samples were collected at the appropriate time points following plasmid administration. Mice were anesthetized IP with Ketamine (60 mg/kg) (Phoenix Scientifics, Inc., St Louis, Mo.). A proparacaine hydrochloride opthalmic solution (Solvay Animal Health Inc., Mendota Heights, Minn.) was applied to the eye. The blood was collected in microtainer serum separator tubes (Becton Dickinson, Franklin Lakes, N.J.) and allowed to clot for 15-30 minutes before centrifuging at 7000 rpm for 5 minutes. Serum levels of SEAP were determined using a chemiluminescence asssay (Tropix, Bedford, Mass.) following the manufacturers instructions.

Through the proper choice of DNA formulation and route of administration, immune responses above and beyond those seen with ‘naked’ DNA may be obtained. These ‘DNA formulations’ have a number of benefits, namely safety, cost, and ease of use, over other strategies such as cytokine augmentation.

### EXAMPLE II

### Effects of Polymer Type and Concentration on Reporter Gene Expression

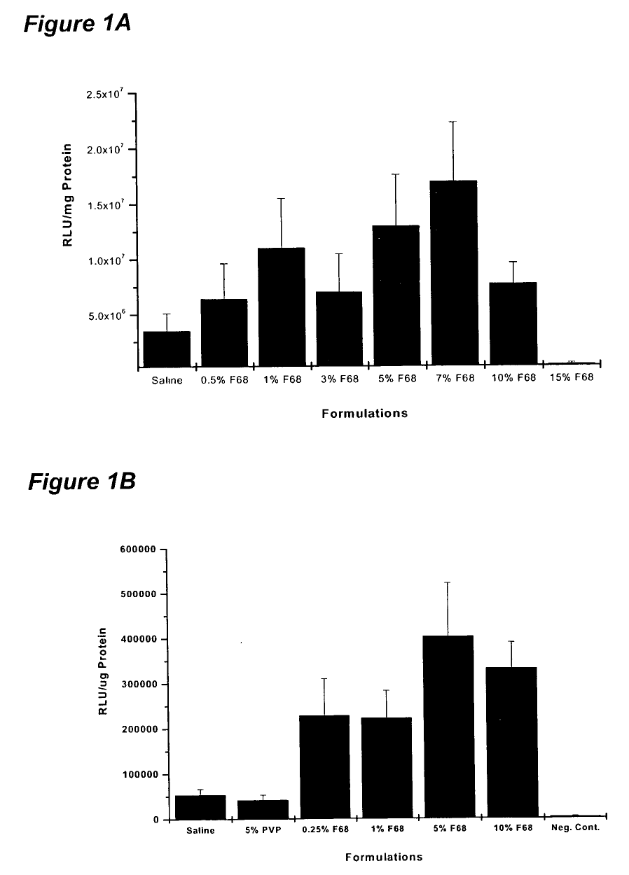

FIG. 1 shows the effect of polymer concentration on the luciferase reporter gene expression in CD-1 mice after IM injection of pDNA in poloxamer 188 formulations. A and B were parallel experiments with 30 micrograms DNA and 10 micrograms DNA/muscle, respectively. For FIG. 1A, results are reported as mean±SEM (n=10) for 10 micrograms pLC0888 in 10 microliters formulation injected into tibialis of CD-1 mice. Harvested at day 7 (n=0). FIG. 1B show the results of 30 micrograms pLC0888 in 10 microliters formulation injected into tibialis and harvested at day 3 (n=10). The results show that poloxamer 188 significantly enhanced gene expression at concentrations ranging from 0.25% to 10%.

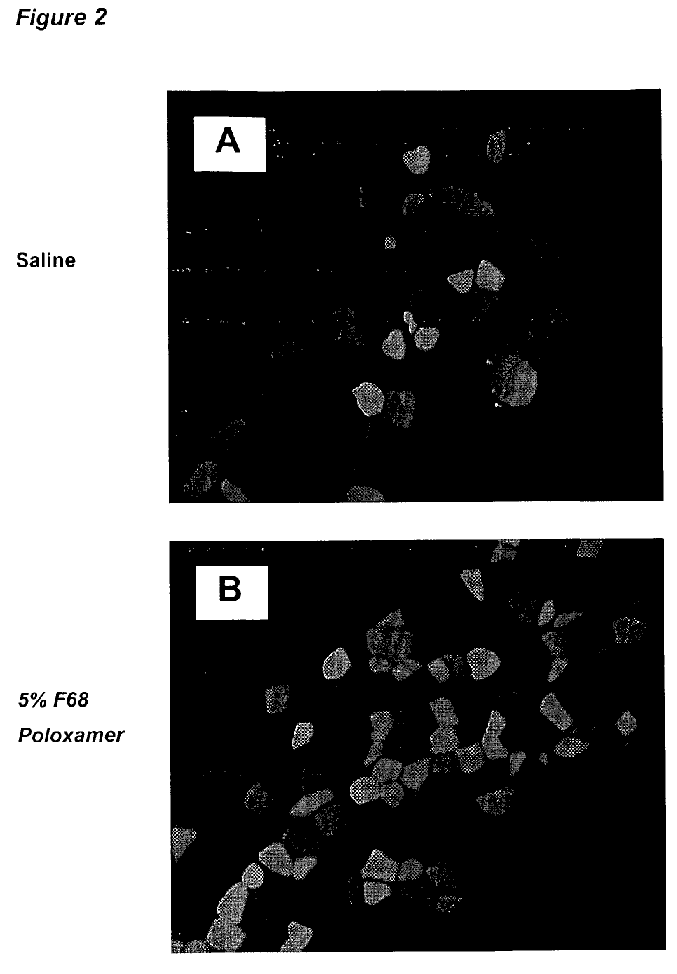

FIG. 2 shows the histology of mice tibialis muscles for Green Fluorescent Protein (GFP) at day 5 after IM injection pDNA in A) saline and B) 5% poloxamer 188 in saline. pDNA injected per muscle was 10 micrograms/10 microliter for a total of 20 microliters for each animal. Expression was enhanced with the poloxamer formulation.

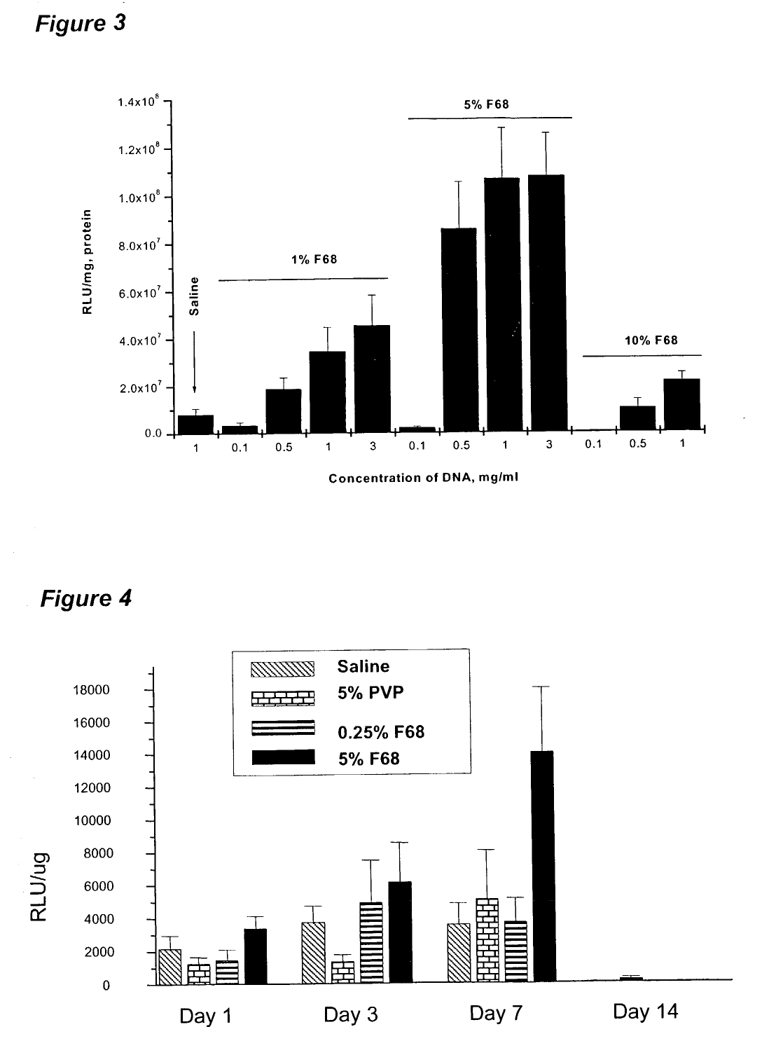

FIG. 3 shows the dose-response of luciferase expression to amount of DNA injected. pLC0888 at concentrations ranging from 0.1 to 3 mg/ml was formulated in concentrations of poloxamer 188 ranging from 1-10% and 10 microliters of formulation was injected into tibialis of CD-1 mice with harvest at day 7 (n=10) after injection. Results are reported as mean±SEM (n=10). Although expression increased continuously with DNA dose at 1% poloxamer, maximum expression was obtained with a DNA concentration of 1 mg/ml in 5% poloxamer.

FIG. 4 shows the time-course of luciferase expression at day 7 after injection of CMV-luciferase encoding plasmid (30 μg of DNA/muscle) formulated in saline, 5% PVP, 0.25% poloxamer 188 (F68) and 5% poloxamer 188 (F68). Plasmid DNA pLC0888 at 3 mg/ml injected in the tibialis muscle at 10 microliters/muscle with a total of 20 microliters per animal. Results are reported as mean±SEM (n=10).

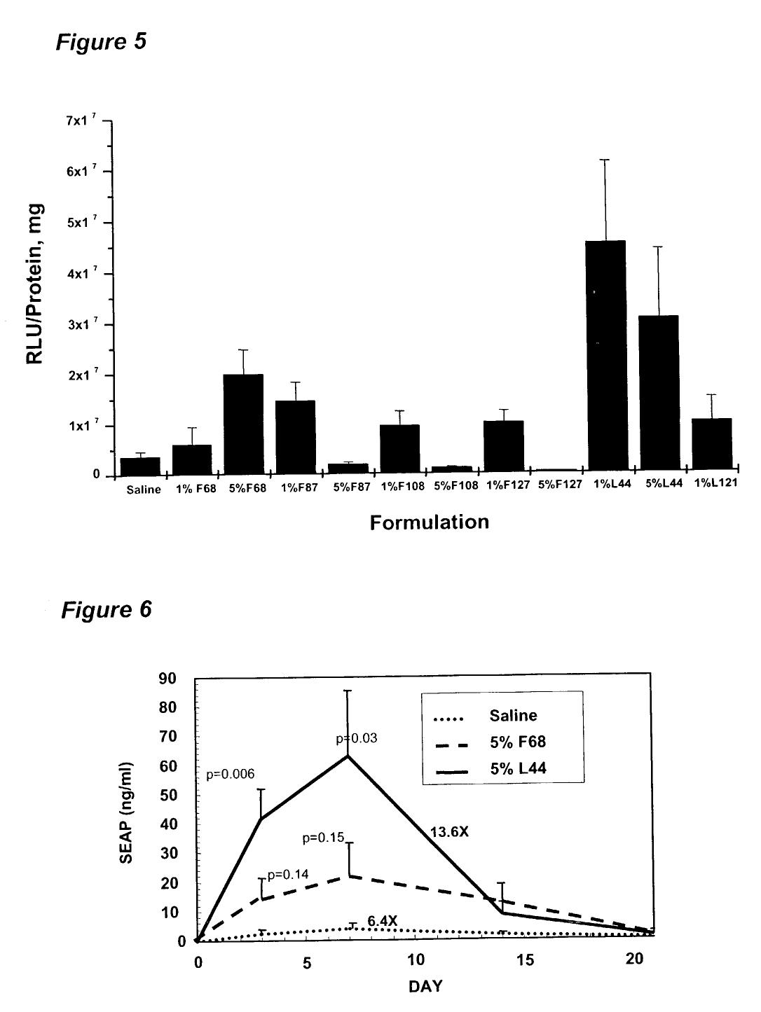

FIG. 5 shows a comparison of selected poloxamer formulations on in vivo gene expression following im injection in mice. Ten (10) microliters of each formulation (1 mg/ml) was injected into CD-1 tibialis and harvested at day 7 (n=10). Significant improvements in gene expression over saline were obtained with 5% F68 and 1% L44. P<0.05. Poloxamer formulations also indicating improvement over saline in this experiment included 5%L44, 1%L121, 1%F87, 1%F108, and 1%F127.

FIG. 6 shows a comparison of 5% L44, 5% F68 and saline with 1 mg/ml SEAP plasmid. Each mouse tibialis muscle recieved 25 microliters, a 50 microgram dose. L44 gave a 17-fold increase (p=0.03) in expression over saline at day 7 and 14-fold increase in area under the curve over 21 days. F68 at 5% was 6-fold better (p=0.15) than saline at day 7 and 6-fold better over 21 days. For both poloxamers, 5% solution were superior to 1% and 10% concentrations.

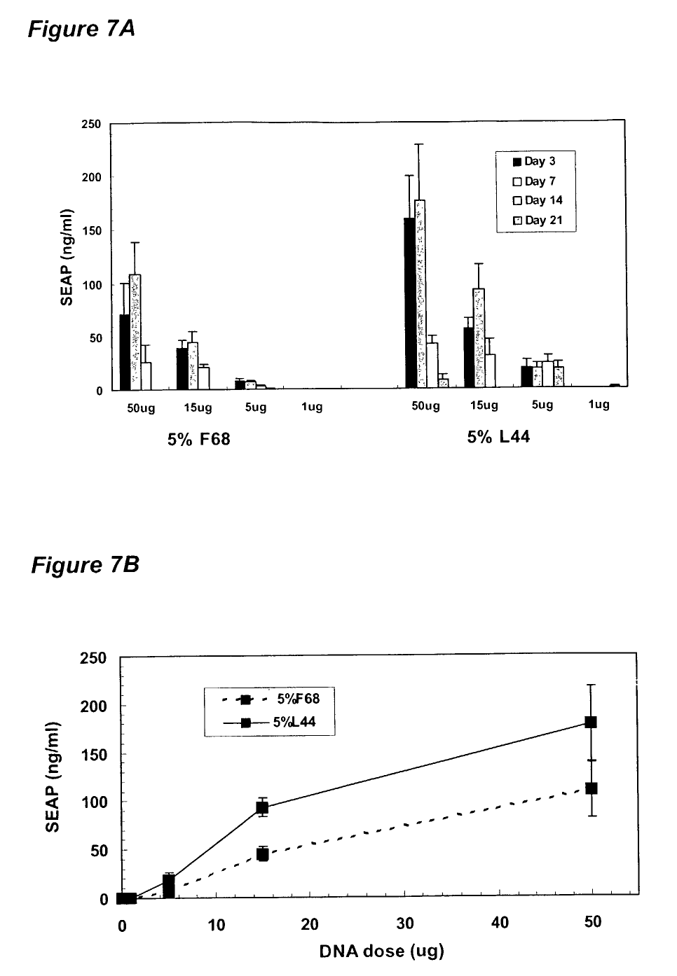

FIGS. 7A and 7B show a dose response of SEAP plasmid 1-50 μg in 5% F68 and 5% L44. Pluronic® L44 was 1.5- to 2.5-fold better than F68 at all concentrations greater than 1 ug.

### EXAMPLE III

### Enhanced Immune Responses Using Polymer Formulations

A number of nucleic acid formulations were screened in either a β-gal or gp100 murine model and the resultant immune response was evaluated by measuring one or more of the following: IgG titers, CTL response, cytokine release from cultured splenocytes, protection from infectious or lethal challenge. Results have indicated that the choice of formulation material, the molar % of formulation material and the route of administration all have profound effects on the resultant immune response. The following polymeric materials have all shown an equivalent response or an enhancement over ‘naked’ DNA in one or more of our assay systems: Pluronic® L121, Tween-20, Tween-80, C12E8, Hydroxypropylcellulose, Carboxymethylcellulose.

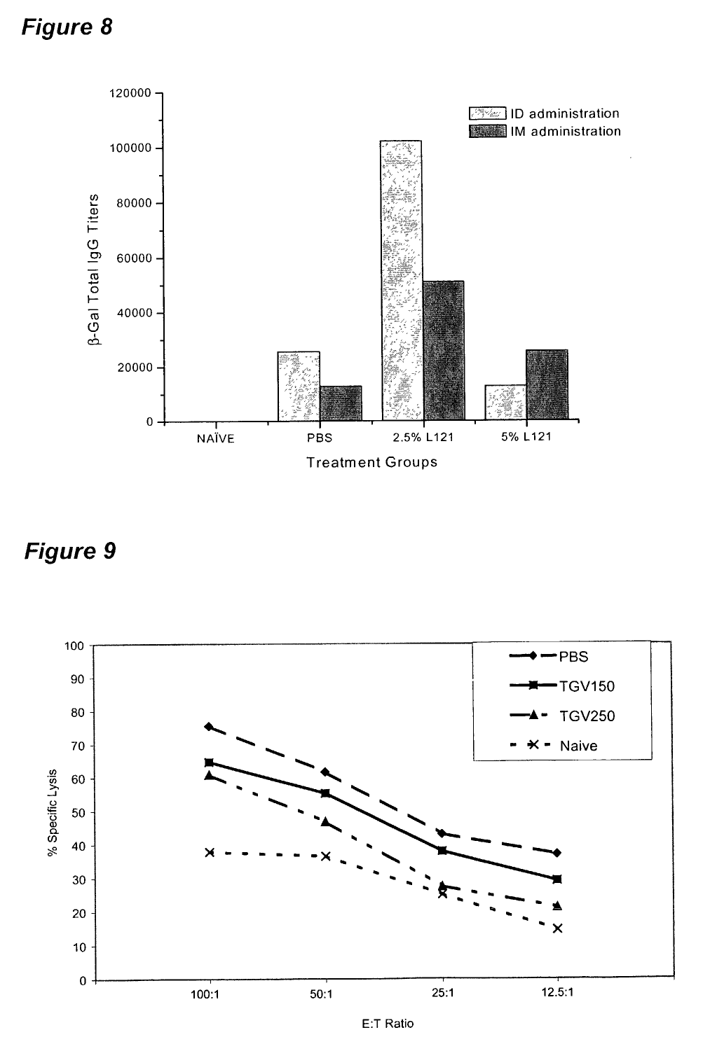

In a typical DNA vaccination experiment 20-25 g Balb/C mice are injected on days 0, 14, and 28 with a formulation containing a PINC polymer and plasmid DNA coding for the model antigen β-galactosidase. On days 28 and 42 the animals are bled and the blood is assayed for total IgG against 1-gal using an ELISA based assay. The humoral results for a typical experiment are shown in FIG. 8. In this experiment DNA is formulated in either 2.5% or 5% Pluronic® L121 in PBS. Naive animals and animals treated with DNA in PBS serve as controls. Mice were injected with 100 microliters of formulated DNA (0.1 mg/ml for 10 μg total dose) either intradermally to a shaved area at the base of the tail or intramuscularly in the tibialis (10 microliters×2 legs) and gastrocnemius (40 microliters×2 legs) muscles. The results demonstrate that antibody levels higher than DNA in PBS can be obtained with DNA formulated in L121. FIG. 8 shows enhanced immune response using poloxamer L121.

In order to look for the presence of a cellular response a CTL assay is typically performed 3-4 weeks following the last immunization. A typical result, depicted in FIG. 9 shows enhanced CTL activity following IM administration of L121/plasmid formulations.

In this experiment animals were again treated intramuscularly as described above on days 0, 14 and 28, however, the formulations now consist of DNA formulated in either 1.5% L121 (TGV150) or 2.5%L121 (TGV250), along with naive and DNA in PBS controls. On or about day 49 spleens were harvested and splenocytes were cultured for a CTL chromium release assay. The results indicate that specific lysis above levels seen in naïve mice can be achieved at all the effector:target ratios tested with DNA formulated in L121. Therefore L121 formulated plasmid can elicit both humoral and cellular responses when used to vaccinate mice with β-gal DNA.

### EXAMPLE IV

### Poloxamer Formulations Confer Long Term Plasmid Stability

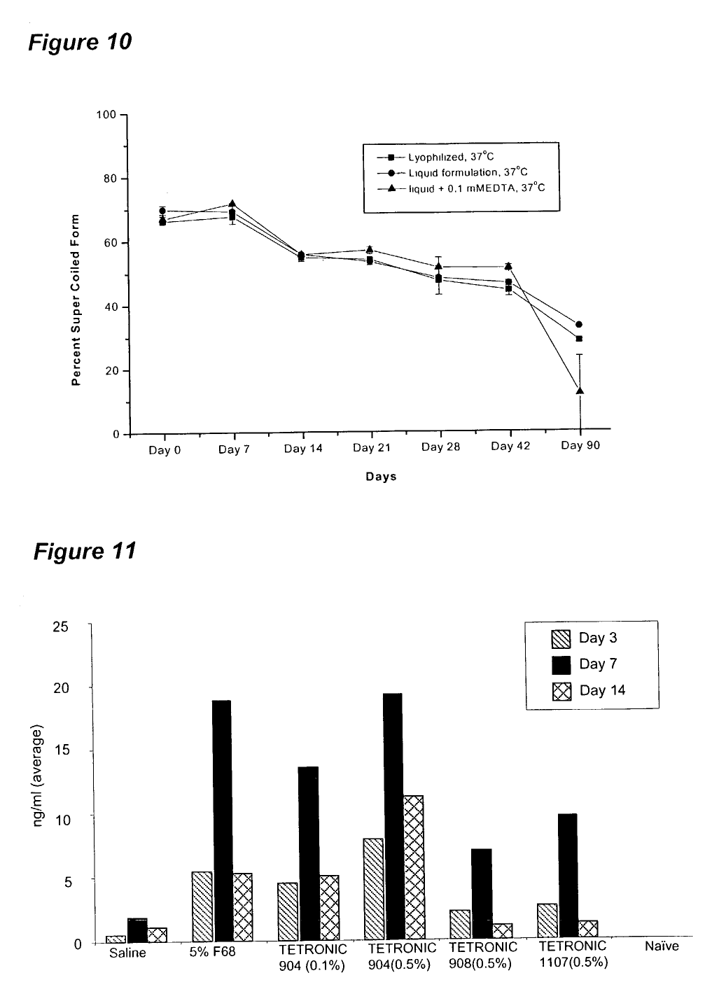

Experiments were undertaken to determine the ability of poloxamer formulations to confer long term stability to plasmid DNA. FIG. 10 shows the stability of human Del-1 encoding plasmid DNA in 5% poloxamer F68 at 37° C. over time. Poloxamer formulations were stabile at 37° C. in either liquid or lyophilized form.

### EXAMPLE V

### Enhancement of Gene Expression Using Poloxamine Formulations

Plasmid DNA encoding SEAP was formulated in various poloxamines obtained from BASF Corporation. Poloxamine concentrations ranging from 0.05% to 5% were tested for TETRONIC® 904, TETRONIC® 908, TETRONIC® 1107 and TETRONIC® 90R4 compared with expression using 5% F68. It was determined that all of these gave increased expression over saline at their ideal concentration. Poloxamine expression was maximal at a concentration of around 0.5% in contrast to poloxamer F68 for which a concentration over 1% is typically required for maximal expression. FIG. 11 shows expression of SEAP from a plasmid concentration of 1 mg/ml comparing different poloxamines with 5% F68 and saline at days 3, 7 and 14 after injection. All of the poloxamines tested resulted in increased expression over saline, particularly at day 7.

### EXAMPLE VI

### Poloxamer Formulations Confer Nuclease Protection

Experiments were undertaken to determine the ability of poly-L-glutamate and Pluronic® F68 to protect plasmid DNA from nuclease digestion. DNase I was obtained from Gibco/BRL (#18068-015). The sodium salt of poly-L-glutamic acid, 2-15 kDa was obtained from Sigma. Pluronic® F68 was obtained from Spectrum. Polymer/DNA 2× stock solutions were prepared (Pluronic® F68=200 micrograms/ml plasmid DNA in 10% F68; Poly-L-glutamate=200 micrograms/ml plasmid DNA in 12 mg/ml sodium poly-L-glutamate). DNase dilutions from 1:10 to 1:10,000 were prepared in 1× DNase buffer. The final reaction mixtures included 25 microliters of the formulation, 15 microliters of water, 5 microliters of 10× DNase buffer and 5 microliters of DNase that were added in the order listed. The reaction mixtures were incubated for 15 minutes at 37° C. and terminated by addition of EDTA prior to gel electrophoresis.

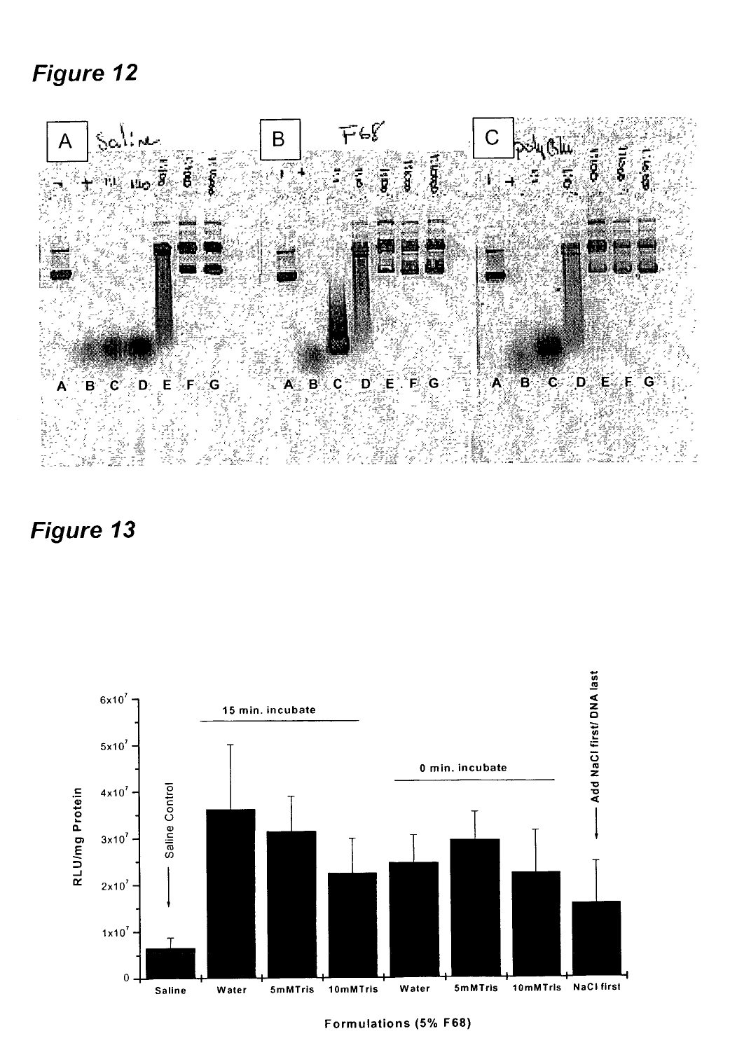

The results of the DNase protection assay are shown in FIG. 12. Panel A represents a DNA in saline formulation; Panel B represents DNA formulated in 5% Pluronic® F68; Panel C represents DNA formulated in 6 mg/ml poly-L-glutamate. Lane A represents the negative control (i.e., plasmid DNA without Dnase); lane B represents the positive control (i.e., plasmid DNA and DNase mixed 1:1); lanes C-G represents the experimental conditions wherein DNA formulated with either saline (Panel A), F68 (Panel B), or poly-glutamate (Panel C) were mixed with DNase diluted 1:1 (lane C); 1:10 (lane D); 1:100 (lane E); 1:1,000 (lane F); and 1:10,000 (lane G). In saline, DNase at 1:100 is able to abolish the lower band of supercoiled plasmid in addition to degradation of the DNA resulting in a smear of different molecular weights on the gel. In contrast, both poly-L-glutamate and Pluronic® F68 were able to confer protection from DNase degradation at 1:100 dilution.

### EXAMPLE VII

### Formulation Process Parameters

Experiments were undertaken to determine whether formulation processes and parameters had an effect on gene expression. The luciferase encoding plasmid pLC0888 was used. Mice were injected with 10 microliters of each formulation into each tibialis muscle, total 20 microliters for each animal following the table below. The muscles were harvested at day 7, collected on dry ice in siliconized eppendorf tubes, lyophilized, and stored at −70° C. until the luciferase assay was performed. Six mice were included in each group.

PLASMID/FORMULATION

A: SALINE (1 MG/ML)
B: 5% f68, 15 MIN INCUBATION. FINAL IN SALINE WITH
0 MM TRIS.
C: 5% F68, 15 MIN INCUBATION. FINAL IN SALINE WITH
5 MM TRIS. ADD TRIS FIRST.
D: 5% F68, 15 MIN INCUBATION. FINAL IN SALINE WITH
10 MM TRIS. ADD TRIS FIRST
E: 5% F68, 0 MIN INCUBATION. FINAL IN SALINE WITH
0 MM TRIS
F: 5% F68, 0 MIN INCUBATION. FINAL IN SALINE WITH
5 MM TRIS. ADD TRIS FIRST.
G: 5% F68, 0 MIN INCUBATION. FINAL IN SALINE WITH
10 MM TRIS. ADD TRIS AND
H. 5% F68, ADD NACL FIRST AND DNA LAST. FINAL IN SALINE.
NO TRIS.

The results of the experiment depicted in FIG. 13 suggest a trend towards greater expression when the polymer and DNA are allowed to interact prior to addition of NaCl. Consequently, the preferred order of addition of constituents to polymer formulations involves mixture of aqueous polymer together with DNA in water or tris up to 10 mM from stock solutions. Adjustment to the desired concentration of the formulated DNA is made with water. Subsequently, NaCl is added to a final 150 mM from a stock solution of 5M NaCl.

### EXAMPLE VIII

### Expression of Therapeutic Genes in Poloxamer Formulations with Electroporation

Del-1 has been recently identified as a factor involved in the development of the vascular system. Del-1 protein and nucleotide sequences encoding human and mouse Del-1 are the subject of U.S. Pat. Nos. 5,877,281 and 5,874,562, incorporated herein by reference in their entirety.

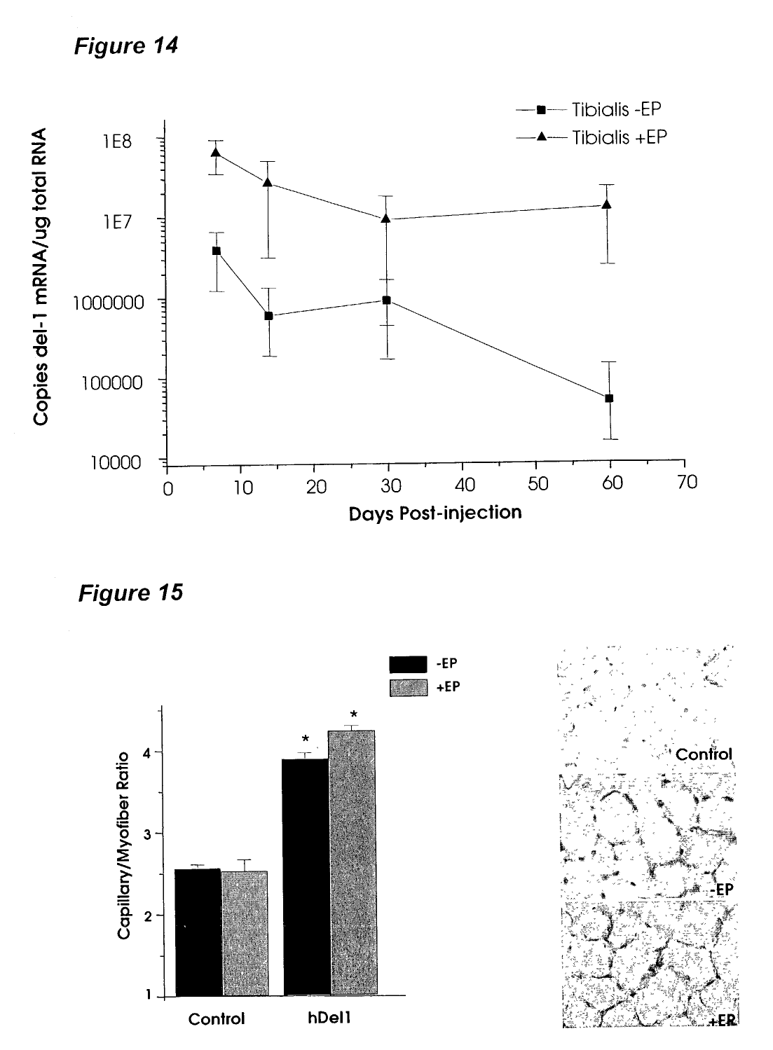

In order to determine the ability of electroporation to affect gene expression of a poloxamer formulated plasmid DNA, the level and duration of mDel-1 expression in tibialis anterior muscles of mice following injection of mDel-1 plasmid DNA formulated in 5% F68 with and without electroporation was determined. Ten micrograms of Del-1 encoding plasmid DNA was injected into the tibialis anterior muscles of CD-1 mice. Injected muscles were harvested at 7, 14, 30 and 60 days post injection and assayed for mDel-1 mRNA by quantitative reverse transciptase PCR (qRT-PCR). Results from this experiment presented on FIG. 14 indicate that expression of mDel-1 decreased at the rate of approximately one log per month when administered without electroporation. Administration of mDel-1 plasmid formulated in 5% F68 in conjunction with electroporation resulted in an approximate two log increase in the level of Del-1 mRNA, and furthermore, appeared to increase the persistence of mDel-1 expression. Data points shown represent the mean+/−SEM for n=5/group/time point.

The biological effect of poloxamer formulated hDel-1 plasmid DNA in normoxic tibialis anterior muscle of mice 7-day post injection with and without electroporation was determined as is shown in FIG. 15. Mice were injected IM into the tibialis anterior with formulated Del-1 or control plasmid followed by use of electroporation to further enhance plasmid uptake (+EP) in half of the injected muscles. Panel A: Results in the bar graph depict capillary density at 7 days post-treatment determined by computer image analysis of CD31 immunostaining. Data show the mean+/−SEM for n=3/group. An asterisk indicates that the groups are different from control (p<0.01). Panel B: Photographs show representative CD-31 immunostaining in muscle cross-sections. A single 10 microgram dose of hDel-1 plasmid formulated in 5%F68 increased capillary:myofiber ratio by approximately 60% (p<0.01). Increasing the level of hDel-1 expression through the use of electroporation did not further increase capillary:myofiber ratio. Although not shown, the effects of human and murine Del-1 in this model were equivalent.

### EXAMPLE IX

### Poloxainer Substantially Increases Expression From Plasmid DNA Delivered by a Percutaneous Route

Experiments were conducted to determine whether a poloxamer formulation would increase gene expression from DNA delivered to muscle via a percutaneous route. Retrograde IV delivery to the left ventricle of pigs was accomplished via placement of a 7F balloon catheter in the mid region of the anterior intraventricular vein and injection of 10 ml of formulated plasmid at a rate of approximately 1 ml/s following inflation of the balloon to occlude venous outflow. Delivery of contrast media or dye via this procedure resulted in localized extravasation of the media into the parenchyma of the left ventricle. Following delivery of the formulated plasmid the inflated balloon was left in place for several minutes (2-10 minutes in pigs depending on the experiment) to increase residence time of the formulation within the tissue. Eight pigs were dosed with formulated plasmid via this route (n=4 with poloxamer formulation, n=4 with cationic lipid formulations). The delivery procedure was well-tolerated in all animals. However, upon harvest at 7 day post administration significant gross pathology was noted in the myocardium of pigs dosed with cationic lipid formulations. Pathology appeared to be more severe in pigs dosed with the 1:3 (−/+) formulation than with the 1:0.5 (−/+) formulation.

Expression of Del-1 mRNA was highest in pigs dosed with the poloxamer 188 formulation and decreased significantly with higher concentrations of cationic lipid as shown in Table 1. Of the delivery modalities and formulations tested, only the poloxamer 188 formulation administered via retrograde IV infusion yielded levels of expression that were comparable to those typically achieved in murine limb muscle following IM injection. Both deliveries via intramyocardial injection and via retrograde IV infusion of a poloxamer formulation appear to be well-tolerated.

TABLE 1
Summary Of Data From Percutaneous Myocardial Delivery Studies
Conducted To Date.

| | | Technical | | | |
|---|---|---|---|---|---|
| Delivery | Formulation | Success¹ | Del-1 mRNA² | | Gross pathology |
| IM catheter | Poloxamer 188 5% | 6/6 | Detectable | (n = 4) | Negative |
| Pericardial | Cationic lipid (1:3, −/+) | 1/3 | <LOQ | (n = 3) | Mild |
| Retrograde IV | Cationic lipid (1:3, −/+) | 2/2 | <LOQ | (n = 2) | Moderate/severe |
| Retrograde IV | Cationic lipid (1:0.5, −/+) | 2/2 | 3997 | (n = 2) | Mild/moderate |
| Retrograde IV | Poloxamer 188 5% | 4/4 | 397279 | (n = 2) | Negative |

¹Technical success is defined as the proportion of delivery procedures that were accomplished without significant problems.
²Copies Del-1 mRNA/μg total RNA, LOQ = 500 copies (500,000-1,000,000 typical level achieved in murine limb muscle.

### EXAMPLE X

### Poloxamer Substantially Increases Expression From Plasmid DNA Delivered by a Percutaneous Route

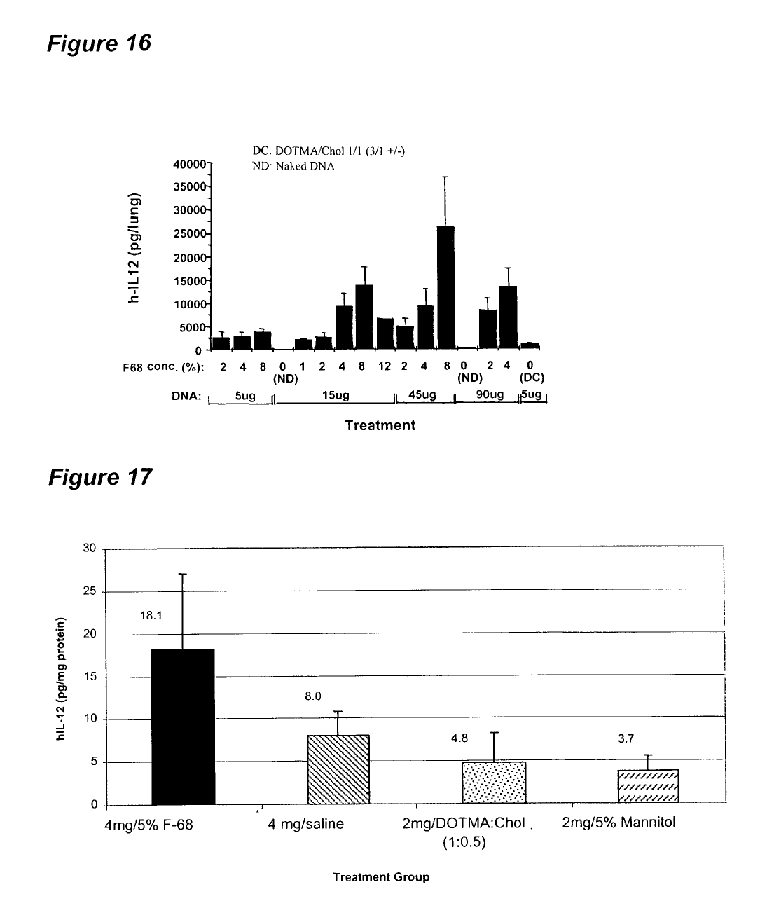

Poloxamer formulations were compared with cationic lipid formulations for percutaneous (intravenous) delivery of plasmid DNA encoding human IL-12 to the lung in a mouse model. The highest poloxamer 188 (F68) expression was observed at 8% (45 ug) and this was 25 fold better than DOTMA/Chol. At the same DNA dose (5 micrograms), poloxamer 188 (F68) (8%) was 3.8 fold better than DOTMA/Chol. Higher delivery with the poloxamer formulations was associated with fewer deaths from metastatic lung tumor. The best expression from 4% F68 (90 ug) was 13 fold better than DC. At the same DNA dose (5 ug), 4% F68 was 2.8 fold better than DC. (FIG. 16)

### EXAMPLE XI

### Poloxamer Substantially Increases Expression From Plasmid DNA Delivered by a Percutaneous Route

Poloxamer formulations were tested for the ability to deliver plasmid DNA encoding cytokines to the liver by hepatic artery. Poloxamer 188 at 5% significantly increased expression of hIL-12 compared with saline, DOTMA/Chol and 5% mannitol. (FIG. 17).

### EXAMPLE XII

### Selected Optimized Poloxamer Formulations for Delivery of Nucleic Acids to the Liver

Anatomy of the Liver for Purposes of Gene Delivery: Blood enters the liver from two sources, the hepatic artery and the hepatic portal vein. The hepatic artery carries oxygenated blood into the sinusoids of the liver. The portal vein carries deoxygenated blood and nutrients from the digestive system into the liver where it also enters the sinusoids. Plates of liver (hepatic) cells line the sinusoids. Blood leaves the liver first through the sinusoid and into the central vein of each lobule before finally leaving the liver through the hepatic vein. Bile produced by the liver cells lining the sinusoids leaves the liver first through the bile canaliculi and ultimately through the bile duct. Thus, compounds delivered to the liver may be considered to be delivered with the normal direction of flow (antegrade) if delivered through the hepatic artery or the hepatic portal vein and against the normal direction of flow (retrograde) if delivered through the hepatic vein or through the bile duct. For site specific delivery of nucleic acids to the liver, various retrograde and antegrade approaches are available and routinely practiced for delivery of conventional drugs and radiography contrast agents. Hepatic artery infusion therapy (HAI) is employed for treatment of liver cancer and slow infusion pumps for this purpose are presently available (for example, Medtronic IsoMed Constant-Flow Infusion System; See, Kemeny, N., et al.; Randomized Study of Hepatic Arterial Infusion (HAI) and Systemic Chemotherapy (SYS) Versus SYS Alone as Adjuvant Therapy After Resection of Hepatic Metastases from Colorectal Cancer. New England Journal of Medicine; 1999).

Retrograde delivery through the bile duct can be accomplished endoscopically as is done with contrast dyes in ERCP procedures (Endoscopic Retrograde Cholangiopancreatograpy) or percutaneously as in PTCA procedures (Percutaneous Transhepatic Cholangiography). For delivery to the liver a catheter is advanced past the cystic duct and into a hepatic duct where the formulated DNA is delivered by either route into the sinusoidal hepatic cells using either supernormal pressure to force the formulation into the intracellular space or by slow infusion. For either pressure delivery or slow infusion, a balloon may inflated and the formulated nucleic acid pushed retrograde through the bile duct and into the bile canaliculi. Delivery of adeno-associated virus (AAV) vectors has been reported to be as effective via ERCP as with direct injection via the portal vein. (Bennett, et al. Effective Delivery of AAV to the Liver via Retrograde Infusion of the Biliary Tree, ASGT Abstract No. 136, 2002)

Delivery of reporter genes to the liver via the portal vein, hepatic vein and bile duct has been reported with even distribution in hepatocytes throughout the entire liver by any of these routes. (Zhang G, et al. Expression of naked plasmid DNA injected into the afferent and efferent vessels of rodent and dog livers. Hum Gene Ther Oct. 10, 1997;8(15):1763-72). Unformulated or “naked” plasmid DNA delivery to the liver via the portal vein either as a bolus injection or by constant infusion has been reported. (Yoshida, et al., Disposition characteristics of plasmid DNA in the single-pass rat liver perfusion system. Pharm Res 1996 April;13(4):599-603). Typically, bolus injection will result in supernormal pressure within the artery, vein or duct. The term “supernormal” in this context means pressure in excess of the normal range of fluid pressure within the respective artery, vein or duct. Supernormal pressure may be expected to force fluid including formulated nucleic acids into the intercellular space between hepatocytes of the liver sinusoids. Where constant infusion is performed, the pressure may be adjusted to either exceed normal pressure or to be similar to normal pressure.

Delivery of cationic lipid complexed with plasmid DNA has been reported for delivery to the liver via the portal vein. However, although cationic lipid delivery via the portal vein significantly enhanced hepatic reporter gene expression, it was associated with significant hepatic toxicity. (Mohr, et al. Cationic liposome-mediated gene delivery to the liver and to hepalocellular carcinomas in mice. Hum Gene Ther May 1, 2001;12(7):799-809).

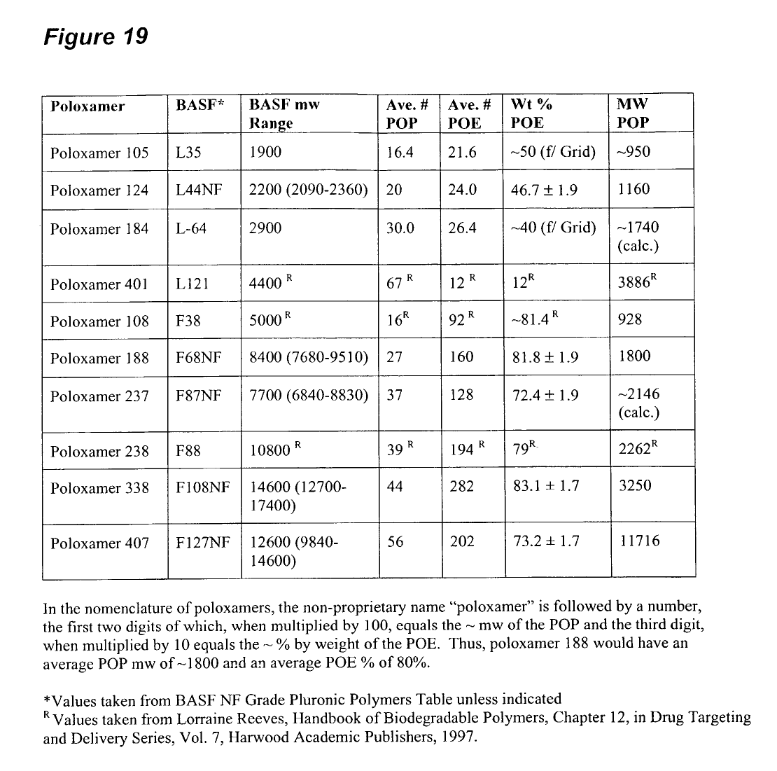

Poloxamer Formulations: Certain poloxamers have been determined by the present inventors to dramatically improve tranfection efficency to the liver while other poloxamers do not present an improvement over saline. Several different poloxamers at several different concentrations were tested in a rat model for enhanced expression from plasmid DNA after either rapid injection into the hepatic artery or by slow infusion. Poloxamer 188 (Pluronic F-68), poloxamer 237 (Pluronic F-87), poloxamer 338 (Pluronic F-108), poloxamer 105 (Pluronic L-35), poloxamer 124 (Pluronic L-44), poloxamer 184 (Pluronic L-64) and poloxamer 401 (Pluronic L-121) were compared to each other, a proprietary poloxamer formulation, SP-1017 (Supratek Pharma; SP-1017 is an 8:1 w/w mixture of poloxamer 407 (Pluronic 127) and polaxamer 181 (Pluronic L-61)), and with saline for determination of optimum poloxamers and concentrations for hepatic delivery. The salient characteristics of these poloxamers are set out in FIG. 19.

Vascular Delivery to the Liver Under Pressure by Hepatic Artery Injection: Hepatic artery injection: Male CD IGS rats (200-250 g) were obtained from Charles River Laboratories, Inc., and housed in the Laboratory Animal Resources vivarium at Valentis. The rats were anesthetized using Isoflurane gas at 2.5% with 1 liter of oxygen per minute. To maintain hydration during surgery, 15 ml/kg of sterile 0.9% isotonic saline was injected subcutaneously in various locations along the back. The surgical site was aseptically prepared and a mid line incision was made to expose the abdominal cavity. The hepatic artery was exposed by gently manually manipulating the abdominal organs. A twenty-eight gauge half-inch needle was used to inject formulated plasmid DNA into the hepatic artery. The area was observed for any hemorrhage prior to suturing the incision. If bleeding persisted, sterile 2×2 gauze and pressure were applied for 1-2 minutess. Chromic gut suture material (4-0) was used to close the rectus abdominus in a continuous suture pattern. The skin incision was closed by autoclips. The animal remained on a water recirculating heating pad until righting reflex returned. Rats were killed 24 hours post-hepatic artery injection and serum and liver were harvested and analyzed for human IL-12 using a commercially available ELISA kit according to the manufacturer's instructions.

Delivery efficiency for different poloxamers was initially tested using direct hepatic artery injection with 2 ml of formulated plasmid encoding human IL-12 formulated in solution with various poloxamers at a final 2 mg/ml plasmid concentration in a 2% poloxamer solution (total of 4 mg per animal). Levels of human IL-12 in the liver and serum were determined by ELISA after 24 hours. As 2% solutions, F-68, F-108, L-35, L-44 and L-64 provided for IL-12 expression that was two to five times the level of expression of plasmid DNA in saline or formulated with SP1017, F-87, or L-121.

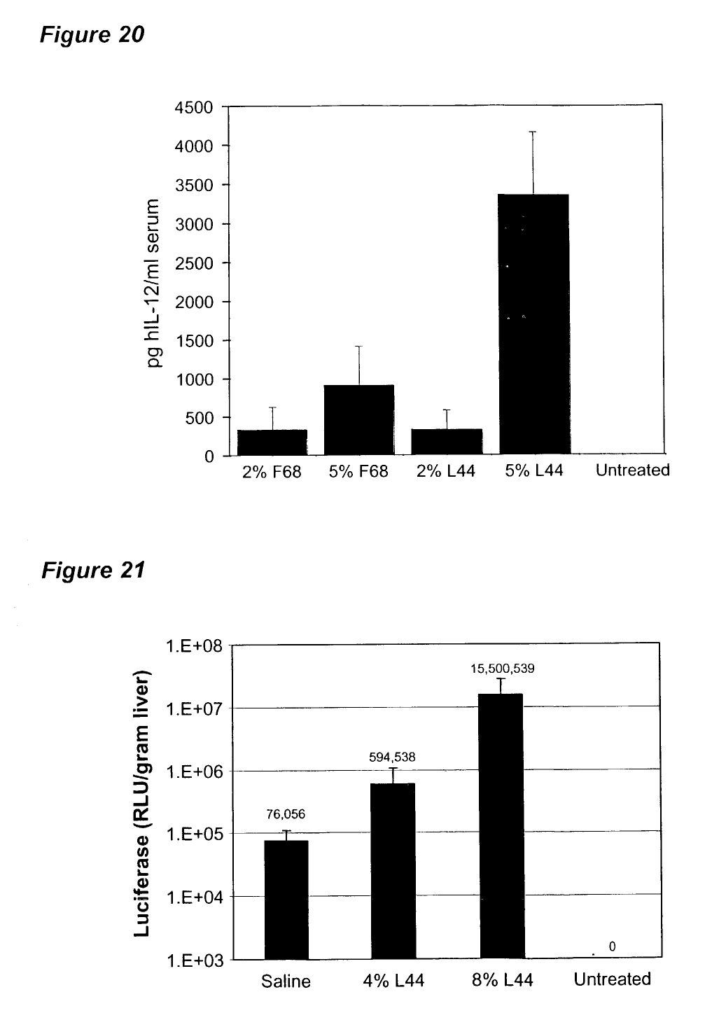

Having selected a subset of poloxamers able to increase gene deliver to the liver, further efforts were directed to selection of desired concentrations. The total plasmid DNA dose was held constant at 4 mg. Rats (n=3) were treated with 2 mL of 2 mg pDNA/mL formulation of human IL12 plasmid (pIN1143) for a total dose of 4 mg plasmid DNA per rat. Improved transgene expression was achieved by increasing the poloxamer concentration from 2% to 5% for F-68 and L-44. However, at 5% L-44 was surprisingly found to be considerably better than F-68. FIG. 20 shows the marked enhancement in serum hIL12 levels with L44 poloxamer when the poloxamer concentration is increased from 2% to 5%. Pluronic L64 at 5% gave expression levels comparable to L44 at 5%.

Slow Infusion of the Hepatic Artery by Catheter: Male CD IGS rats (350-400 g) were obtained from Charles River Laboratories, Inc., and housed in the Laboratory Animal Resources vivarium at Valentis. Rats were anesthetized for hepatic artery catheterization using combination anesthesia [ketamine (73.96 mg/ml), xylazine (3.75 mg/ml), and acepromazine (73 mg/ml)] given at 0.7-1.0 ml/Kg body weight. The surgical site was aseptically prepared and a mid line incision was made to expose the abdominal cavity. The hepatic artery, between the hepatic proper and gastroduodenal artery, was exposed by gentle manual manipulation of the abdominal organs. The distal end was ligated, and the proximal end was temporarily ligated with a loose knot and 5-0 silk. A small hole was cut into the artery and a polyurethane catheter (0.025″×0.40″) was inserted such that the tip of the catheter is located at the junction of the hepatic artery and hepatic proper artery. The catheter was tied in place and the catheter was routed subcutaneously to surface dorsally between the scapula using a #10 trochar and clipped into place. After checking to ensure proper placement, the catheter was flushed with sterile saline and filled with a sterile “lock” solution of heparinized glycerol. A small wire plug was inserted in the catheter to hold the lock solution and to ensure the catheter remained open. The abdominal area was observed for any hemorrhage prior to suturing the incision. If bleeding persisted, sterile 2×2 gauze and pressure were applied for 1-2 minutes. The rectus abdominus was closed in a continuous suture pattern using 4-0 chromic gut suture material, and the skin incision was closed with autoclips. The animal remained on a water recirculating heating pad until righting reflex returned. Formulated luciferase plasmid DNA (pLC0888, 2 mL of 1 mg/mL) was infused through the catheter using a syringe pump at a rate of 100 microliters/minute. Rats were harvested 24 hours post-infusion and liver tissue was snap frozen in liquid nitrogen, homogenized, and assayed for luciferase expression.

FIG. 21 shows luciferase expression in liver tissue of rats treated with plasmid DNA formulated in saline, 4% L44, or 8% L44 by slow infusion through a hepatic artery catheter. 4% L44 and 8% L44 enhanced luciferase expression 7.8-fold and 204-fold, respectively, compared to saline. Luciferase expression with 8% L44 was 26-fold higher than with 4% L44.

One skilled in the art would readily appreciate that the present invention is well adapted to carry out the objects and obtain the ends and advantages mentioned, as well as those inherent therein. The molecular complexes and the methods, procedures, treatments, molecules, specific compounds described herein are presently representative of preferred embodiments are exemplary and are not intended as limitations on the scope of the invention. Changes therein and other uses will occur to those skilled in the art which are encompassed within the spirit of the invention are defined by the scope of the claims.

It will be readily apparent to one skilled in the art that varying substitutions and modifications may be made to the invention disclosed herein without departing from the scope and spirit of the invention.

All patents and publications mentioned in the specification are indicative of the levels of those skilled in the art to which the invention pertains. All patents and publications are herein incorporated by reference to the same extent as if each individual publication was specifically and individually indicated to be incorporated by reference.

Those references not previously incorporated herein by reference, including both patent and non-patent references, are expressly incorporated herein by reference for all purposes. Other embodiments are within the following claims.

1. A composition for delivery of a nucleic acid molecule to a cell, comprising a non-condensing polymer selected from the group consisting of Poloxamer 188, Poloxamer 237, Poloxamer 338, Poloxamer 124, Poloxamer 401, Tetronic 904, Tetronic 908, Tetronic 1107 and Tetronic 90R4 and (b) a nucleic acid molecule that comprises a sequence encoding a gene product.

2. The composition of claim 1, wherein said gene product is a therapeutic polypeptide or protein.

3. The composition of claim 1, wherein said polymer is bound with a targeting ligand.

4. The composition of claim 3, wherein said targeting ligand is an antibody.

5. The composition of claim 1, wherein said nucleic acid is selected from the group consisting of condensed nucleic acid, nucleic acid formulated with cationic lipids, nucleic acid formulated with peptides or cationic polymers.

6. The composition of claim 1, wherein said polymer is present at a concentration of 0.05% to 10%.

7. The composition of claim 6, wherein said polymer is present at a concentration of about 10% (w/v) or less.

8. The composition of claim 7, wherein said polymer is present at a concentration of 8.0% or less.

9. The composition of claim 8, wherein said polymer is present at a concentration of 5% or less.

10. The composition of claim 3, wherein said targeting ligand is bound to said polymer by non-covalent interactions.

11. The composition of claim 3, wherein said targeting ligand is bound to said polymer by covalent bonding.

12. The composition of claim 1, wherein said nucleic acid molecule is one or more plasmids with a eukaryotic promoter which expresses one or more therapeutic molecules.

13. The composition of claim 1, wherein said nucleic acid molecule comprises RNA.

14. A method of administering to a mammal a composition for delivery of a nucleic acid molecule to a cell, comprising the step of introducing a composition of claim 1 into a tissue of a mammal.

15. The method of claim 14, wherein said method results in an antibody response.

16. The method of claim 14, wherein said method induces an immune response.

17. The method of claim 14, wherein said step of introducing said composition into a tissue of a mammal is by injection.

18. The method of claim 14, wherein said tissue is muscle.

19. The method of claim 17, wherein said tissue is a tumor.

20. A method for delivering a nucleic acid molecule to an organism comprising the step of providing a composition of claim 1 to the cells of said organism by use of a device configured and arranged to cause pulse voltage delivery of said composition.

21. The method of claim 20, wherein said organism is a mammal.

22. The method of claim 20, wherein said mammal is a human.

23. The method of claim 20, wherein said device for delivering is an electroporation device that delivers said composition to said cell by pulse voltage.

24. A kit comprising a container for providing a composition of claim 1 and either (i) a pulse voltage device for delivering said composition to cells of an organism, wherein said pulse voltage device is capable of being combined with said container, or (ii) instructions explaining how to deliver said composition with said pulse voltage device.

25. A method of treating a mammal suffering from cancer or an infectious disease, comprising the step of providing a composition of claim 1 to cells of said mammal by use of a device configured and arranged to pulse voltage delivery of said composition to cells of said mammal, wherein said molecule encodes a cancer antigen or an antigen for said infectious disease.

26. The method of claim 25, wherein said cancer antigen is MAGE 1, and said cancer is melanoma.

27. The method of claim 25, wherein said infectious disease antigen is HBV core antigen, and said infectious disease is chronic hepatitis.

28. A composition for delivery of a nucleic acid molecule to a cell, comprising (a) a Poloxamer selected from the group consisting of Poloxamer 188, Poloxamer 237, Poloxamer 338, Poloxamer 124 and Poloxamer 401 and (b) a nucleic acid molecule that comprises a sequence encoding a gene product.

29. The composition of claim 28, wherein said Poloxamer is Poloxamer 188.

30. The composition of claim 29, wherein said Poloxamer 188 is present at a concentration of 0.25% to 10% (w/v).

31. The composition of claim 30, wherein said Poloxamer 188 is present at a concentration of about 5% to about 8.0% (w/v).

32. The composition of claim 28, wherein said Poloxamer 188 is present at a concentration of about 8.0% (w/v) or less.

33. The composition of claim 33, wherein said Poloxamer 188 is present at a concentration of about 5% (w/v) or less.

34. The composition of claim 28, wherein said Poloxamer is Poloxamer 237.

35. The composition of claim 34, wherein the Poloxamer 237 is present in a concentration of about 1% (w/v) or less.

36. The composition of claim 28, wherein said Poloxamer is Poloxamer 338.

37. The composition of claim 36, wherein the Poloxamer 338 is present in a concentration of about 1% (w/v) or less.

38. The composition of claim 28, wherein said Poloxamer is Poloxamer 124.

39. The composition of claim 38, wherein the Poloxamer 124 is present in a concentration of about 5% (w/v) or less.

40. The composition of claim 28, wherein said Poloxamer is Poloxamer 401.

41. The composition of claim 40, wherein the Poloxamer 401 is present in a concentration of 5% (w/v) or less.

42. The composition of claim 41, wherein the Poloxamer 401 is present in a concentration of about 2.5% (w/v) or less.

43. A method of administering to a mammal a composition for delivery of a nucleic acid molecule to a cell, comprising the step of introducing a composition of claim 28 into a tissue of a mammal.

44. A composition for delivery of a nucleic acid molecule to a cell, comprising (a) a Poloxamine selected from the group consisting of Tetronic 904, Tetronic 908, Tetronic 1107 and Tetronic 90R4 and (b) a nucleic acid molecule that comprises a sequence encoding a gene product.

45. The composition of claim 44, wherein said Poloxamine is present at a concentration of 0.05% to 5% (w/v).

46. The composition of claim 45, wherein said Poloxamine is Tetronic 908 and is present at a concentration of about 1% to about 5% (w/v).

47. The composition of claim 44, wherein said Poloxamine is Tetronic 904.

48. The composition of claim 47, wherein the Tetronic 904 is present in a concentration of about 0.5% (w/v) or less.

49. The composition of claim 48, wherein the Tetronic 904 is present in a concentration of about 0.1% (w/v) or less.

50. A method of administering to a mammal a composition for delivery of a nucleic acid molecule to a cell, comprising the step of introducing a composition of claim 44 into a tissue of a mammal.

51. A composition for delivery of a nucleic acid molecule to a cell within a liver, comprising a poloxamer and a nucleic acid molecule that encodes a gene product, wherein the composition is administered locally to the liver.

52. The composition of claim 51, wherein said poloxamer is selected from the group consisting of poloxamer 124 (L-44), poloxamer 188 (F-68), poloxamer 338 (F108), poloxamer 105 (L-35), and poloxamer 124 (L-64).

53. The composition of claim 52, wherein said poloxamer is present at a concentration of about 10% (w/v) or less.

54. The composition of claim 51, wherein said poloxamer is selected from the group consisting of poloxamer 124 (L-44) and poloxamer 124 (L-64).

55. The composition of claim 54, wherein said poloxamer is present at a concentration of between about 2% to about 10% (w/v).

56. The composition of claim 55, wherein said poloxamer is present at a concentration of between about 4% and about 8.0%.

57. A composition for delivery of a nucleic acid molecule to a cell within a liver, consisting essentially of a poloxamer and a nucleic acid molecule that encodes a gene product, wherein the composition is administered locally to the liver.

58. The composition of claim 57, wherein said poloxamer is selected from the group consisting of poloxamer 124 (L-44) and poloxamer 124 (L-64) and wherein said poloxamer is present at a concentration of between about 2% to about 10% (w/v).

59. A method of administering a nucleic acid molecule to a cell within a liver, comprising the step of introducing the composition of claim 51 into the liver by injection into a hepatic vein, a hepatic portal vein, a hepatic artery or a bile duct.

60. The method of claim 59, wherein said composition is administered by injection under supernormal pressure.

61. The method of claim 59, wherein said composition is administered by slow infusion injection.
